SEQUENCE LISTING (8 sequences)

SEQ ID NO: 1 - protein, 244 aa, Homo sapiens
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 2 - DNA, 4629 bp, Homo sapiens
aggctgttgaggctgggccatctcctcctcacttccattctgactgcagtctgtggttctgattccataccagaggagacgggatttcaccatgttgtccaggctggtctgaaactcctgacatcagggctcaggatgctgttgctgggagctgttctactgctattagctctgcccggtcatgaccaggaaaccacgactcaagggcccggagtcctgcttcccctgcccaagggggcctgcacaggttggatggcgggcatcccagggcatccgggccataatggggccccaggccgtgatggcagagatggcacccctggtgagaagggtgagaaaggagatccaggtcttattggtcctaagggagacatcggtgaaaccggagtacccggggctgaaggtccccgaggctttccgggaatccaaggcaggaaaggagaacctggagaaggtgcctatgtataccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactgatcaccactaactcagagcctcctccaggccaaacagccccaaagtcaattaaaggctttcagtacggttaggaagttgattattatttagttggaggcctttagatattattcattcatttactcattcatttattcattcattcatcgagtaactttaaaaaaatcatatgctatgttcccagtcctggggagcttcacaaacatgaccagataactgactagaaagaagtagttgacagtgctattttgtgcccactgtctctcctgatgctcatatcaatcctataaggcacagggaacaagcattctcctgtttttacagattgtatcctgaggctgagagagttaagtgaatgtctaaggtcacacagtattaagtgacagtgctagaaatcaaacccagagctgtggactttgttcactagactgtgcccttttatagaggtacatgttctctttggagtgttggtaggtgtctgtttcccacctcacctgagagccattgaatttgccttcctcatgaattaaaacctcccccaagcagagcttcctcagagaaagtggttctatgatgacgtcctgtcttggaaggactactactcaatggcccctgcactactctacttcctcttacctatgtcccttctcatgcctttccctccaacggggaaagccaactccatctctaagtgccgaactcatccctgttcctcaaggccacctggccaggagcttctctgatgtgatatccactttttttttttttgagatggagtctcactctgtcacccaggctggagtacagtgacacgacctcggctcactgcagcctccttctcctgggtccaagcaattattgtgcctcagcctcccgagtagctgagacttcaggtgcattccaccacacatggctaatttttgtatttttagtagaaatggggtttcgtcatgttggccaggctggtctcgaactcctggcctaggtgatccacccgcctcgacctcccaaagtgctgggattacaggcatgagccaccatgcccagtcgatatctcactttttattttgccatggatgagagtcctgggtgtgaggaacacctcccaccaggctagaggcaactgcccaggaaggactgtgcttccgtcacctctaaatcccttgcagatccttgataaatgcctcatgaagaccaatctcttgaatcccatatctacccagaattaactccattccagtctctgcatgtaatcagttttatccacagaaacattttcattttaggaaatccctggttttaagtatcaatccttgttcagctggacaatatgaatcttttccactgaagttagggatgactgtgattttcagaacacgtccagaatttttcatcaagaaggtagcttgagcctgaaatgcaaaacccatggaggaattctgaagccattgtctccttgagtaccaacagggtcagggaagactgggcctcctgaatttattattgttctttaagaattacaggttgaggtagttgatggtggtaaacattctctcaggagacaataactccagtgatgttcttcaaagattttagcaaaaacagagtaaatagcattctctatcaatatataaatttaaaaaactatctttttgcttacagttttaaattctgaacaattctctcttatatgtgtattgctaatcattaaggtattattttttccacatataaagctttgtctttttgttgttgttgttgtttttaagatggagtttccctctgttgccaggctagagtgcagtggcatgatctcggcttactgcaacctttgcctcccaggttcaagcgattcttctgcctcagcctcccgagtagctgggaccacaggtgcctaccaccatgccaggctaatttttgtatttttagtaaagacagggtttcaccatattggccaggctggtctcgaactcctgaccttgtgatctgcccgcctccatttttgttgttattttttgagaaagatagatatgaggtttagagagggatgaagaggtgagagtaagccttgtgttagtcagaactctgtgttgtgaatgtcattcacaacagaaaacccaaaatattatgcaaactactgtaagcaagaaaaataaaggaaaaatggaaacatttattcctttgcataatagaaattaccagagttgttctgtctttagataaggtttgaaccaaagctcaaaacaatcaagacccttttctgtatgtccttctgttctgccttccgcagtgtaggctttaccctcaggtgctacacagtatagttctagggtttccctcccgatatcaaaaagactgtggcctgcccagctctcgtatccccaagccacaccatctggctaaatggacatcatgttttctggtgatgcccaaagaggagagaggaagctctctttcccagatgccccagcaagtgtaaccttgcatctcattgctctggctgagttgtgtgcctgtttctgaccaatcactgagtcaggaggatgaaatattcatattgacttaattgcagcttaagttaggggtatgtagaggtattttccctaaagcaaaattgggacactgttatcagaaataggagagtggatgatagatgcaaaataatacctgtccacaacaaactcttaatgctgtgtttgagctttcatgagtttcccagagagacatagctggaaaattcctattgattttctctaaaatttcaacaagtagctaaagtctggctatgctcacagtctcacatctggttggggtgggctccttacagaacacgctttcacagttaccctaaactctctggggcagggttattcctttgtggaaccagaggcacagagagagtcaactgaggccaaaagaggcctgagagaaactgaggtcaagatttcaggattaatggtcctgtgatgctttgaagtacaattgtggatttgtccaattctctttagttctgtcagcttttgcttcatatattttagcgctctattattagatatatacatgtttagtattatgtcttattggtgcatttactctcttatcattatgtaatgtccttctttatctgtgataattttctgtgttctgaagtctactttgtctaaaaataacatacgcactcaacttccttttctttcttccttcctttctttcttccttcctttctttctctctctctctctttccttccttccttcctccttttctttctctctctctctctctctctttttttgacagactctcgttctgtggccctggctggagttcagtggtgtgatcttggctcactgctacctctaccatgagcaattctcctgcctcagcctcccaagtagctggaactacaggctcatgccactgcgcccagctaatttttgtatttttcgtagagacggggtttcaccacattcgtcaggttggtttcaaactcctgactttgtgatccacccgcctcggcctcccaaagtgctgggattacaggcatgagccatcacacctggtcaactttcttttgattagtgtttttgtggtatatctttttccatcatgttactttaaatatatctatattattgtatttaaaatgtgtttcttacagactgcatgtagttgggtataatttttatccagtctaaaaatatctgtcttttaattggtgtttagacaatttatatttaataaaattgttgaatttaa

SEQ ID NO: 3 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 4 - DNA, 2024 bp, Homo sapiens
aaggcaagagatctaggacttctagcccctgaactttcagccgaatacatcttttccaaaggagtgaattcaggcccttgtatcactggcagcaggacgtgaccatggagaagctgttgtgtttcttggtcttgaccagcctctctcatgcttttggccagacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctgaggcccagctgtgggtcctgaaggtacctcccggttttttacaccgcatgggccccacgtctctgtctctggtacctcccgcttttttacactgcatggttcccacgtctctgtctctgggcctttgttcccctatatgcattgcaggcctgctccaccctcctcagcgcctgagaatggaggtaaagtgtctggtctgggagctcgttaactatgctgggaaacggtccaaaagaatcagaatttgaggtgttttgttttcatttttatttcaagttggacagatcttggagataatttcttacctcacatagatgagaaaactaacacccagaaaggagaaatgatgttataaaaaactcataaggcaagagctgagaaggaagcgctgatcttctatttaattccccacccatgacccccagaaagcaggagggcattgcccacattcacagggctcttcagtctcagaatcaggacactggccaggtgtctggtttgggtccagagtgctcatcatcatgtcatagaactgctgggcccaggtctcctgaaatgggaagcccagcaataccacgcagtccctccactttctcaaagcacactggaaaggccattagaattgccccagcagagcagatctgctttttttccagagcaaaatgaagcactaggtataaatatgttgttactgccaagaacttaaatgactggtttttgtttgcttgcagtgctttcttaattttatggctcttctgggaaactcctccccttttccacacgaaccttgtggggctgtgaattctttcttcatccccgcattcccaatatacccaggccacaagagtggacgtgaaccacagggtgtcctgtcagaggagcccatctcccatctccccagctccctatctggaggatagttggatagttacgtgttcctagcaggaccaactacagtcttcccaaggattgagttatggactttgggagtgagacatcttcttgctgctggatttccaagctgagaggacgtgaacctgggaccaccagtagccatcttgtttgccacatggagagagactgtgaggacagaagccaaactggaagtggaggagccaagggattgacaaacaacagagccttgaccacgtggagtctctgaatcagccttgtctggaaccagatctacacctggactgcccaggtctataagccaataaagcccctgtttacttgaaaaaaaaaa

SEQ ID NO: 5 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 6 - DNA, 469 bp, Homo sapiens
agccctccaggacaggctgcatcagaagaggccatcaagcagatcactgtccttctgccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactagacgcagcccgcaggcagccccacacccgccgcctcctgcaccgagagagatggaataaagcccttgaaccagcaaaa

SEQ ID NO: 7 - protein, 167 aa, Homo sapiens
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 8 - DNA, 3444 bp, Homo sapiens
gtaggaatcgcagcgccagcggttgcaaggcccaagaagcccatcctgggaaggaaaatgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgctgaggccttgaaggtcactcttcctgcaaggactacgttaagggaaggaactctggcttccaggtatctccaggattgaagagcattgcatggacaccccttatccaggactctgtcaatttccctgactcctctaagccactcttccaaaggcataagaccctaagcctccttttgcttgaaaccaaagatatatacacaggatcctattctcaccaggaagggggtccacccagcaaagagtgggctgcatctgggattcccaccaaggtcttcagccatcaacaagagttgtcttgtcccctcttgacccatctccccctcactgaatgcctcaatgtgaccaggggtgatttcagagagggcagaggggtaggcagagcctttggatgaccagaacaaggttccctctgagaattccaaggagttccatgaagaccacatccacacacgcaggaactcccagcaacacaagctggaagcacatgtttatttattctgcattttattctggatggatttgaagcaaagcaccagcttctccaggctctttggggtcagccagggccaggggtctccctggagtgcagtttccaatcccatagatgggtctggctgagctgaacccattttgagtgactcgagggttgggttcatctgagcaagagctggcaaaggtggctctccagttagttctctcgtaactggtttcatttctactgtgactgatgttacatcacagtgtttgcaatggtgttgccctgagtggatctccaaggaccaggttattttaaaaagatttgttttgtcaagtgtcatatgtaggtgtctgcacccaggggtggggaatgtttgggcagaagggagaaggatctagaatgtgttttctgaataacatttgtgtggtgggttctttggaaggagtgagatcattttcttatcttctgcaattgcttaggatgtttttcatgaaaatagctctttcaggggggttgtgaggcctggccaggcaccccctggagagaagtttctggccctggctgaccccaaagagcctggagaagctgatgctttgcttcaaatccatccagaataaaacgcaaagggctgaaagccatttgttggggcagtggtaagctctggctttctccgactgctagggagtggtctttcctatcatggagtgacggtcccacactggtgactgcgatcttcagagcaggggtccttggtgtgaccctctgaatggtccagggttgatcacactctgggtttattacatggcagtgttcctatttggggcttgcatgccaaattgtagttcttgtctgattggctcacccaagcaaggccaaaattaccaaaaatcttggggggtttttactccagtggtgaagaaaactcctttagcaggtggtcctgagacctgacaagcactgctaggcgagtgccaggactccccaggccaggccaccaggatggcccttcccactggaggtcacattcaggaagatgaaagaggaggtttggggtctgccaccatcctgctgctgtgtttttgctatcacacagtgggtggtggatctgtccaaggaaacttgaatcaaagcagttaactttaagactgagcacctgcttcatgctcagccctgactggtgctataggctggagaagctcacccaataaacattaagattgaggcctgccctcagggatcttgcattcccagtggtcaaaccgcactcacccatgtgccaaggtggggtatttaccacagcagctgaacagccaaatgcatggtgcagttgacagcaggtgggaaatggtatgagctgaggggggccgtgcccaggggcccacagggaaccctgcttgcactttgtaacatgtttacttttcagggcatcttagcttctattatagccacatccctttgaaacaagataactgagaatttaaaaataagaaaatacataagaccataacagccaacaggtggcaggaccaggactatagcccaggtcctctgatacccagagcattacgtgagccaggtaatgagggactggaaccagggagaccgagcgctttctggaaaagaggagtttcgaggtagagtttgaaggaggtgagggatgtgaattgcctgcagagagaagcctgttttgttggaaggtttggtgtgtggagatgcagaggtaaaagtgtgagcagtgagttacagcgagaggcagagaaagaagagacaggagggcaagggccatgctgaagggaccttgaagggtaaagaagtttgatattaaaggagttaagagtagcaagttctagagaagaggctggtgctgtggccagggtgagagctgctctggaaaatgtgacccagatcctcacaaccacctaatcaggctgaggtgtcttaagccttttgctcacaaaacctggcacaatggctaattcccagagtgtgaaacttcctaagtataaatggttgtctgtttttgtaacttaaaaaaaaaaaaaaaagtttggccgggtgcggtggctcacgcctgtaatcccagcactttgggaggccaaggtggggggatcacaaggtcactagatggcgagcatcctggccaacatggtgaaaccccgtctctactaaaaacacaaaagttagctgagcgtggtggcgggcgcctgtagtcccagccactcgggaggctgagacaggagaatcgcttaaacctgggaggcggagagtacagtgagccaagatcgcgccactgcactccggcctgatgacagagcgagattccgtcttaaaaaaaaaaaaaaaaaagtttgtttttaaaaaaatctaaataaaataactttgccccctgcaaaaaaaaaaaaaaaaa

(19) United States
(12) Patent Application Publication
(10) Pub. No.: US 2016/0231334 A1
(43) Pub. Date: Aug. 11, 2016

(54) Compositions and Methods for Evaluating Metabolic Syndrome and Related Diseases

(71) Applicant: THE FORSYTH INSTITUTE, Cambridge, MA (US)

(72) Inventor: Max J. Goodson, Cambridge, MA (US)

(73) Assignee: The Forsyth Institute, Cambridge, MA (US)

(21) Appl. No.: 15/029,980

(22) PCT Filed: Oct. 28, 2014
(86) PCT No.: PCT/US2014/062665

§ 371 Date: Apr. 15, 2016

Related U.S. Application Data
(60) Provisional application No. 61/896,446, filed on Oct. 28, 2013.

(51) Int. Cl. G01N 33/68

(52) U.S. Cl. CPC G01N 33/6893; G01N 2800/04; G01N 2800/044; G01N 2800/60; G01N 2800/50

(57) ABSTRACT

The invention features panels of salivary biomarkers useful for identifying subjects having or at risk of developing a metabolic disease, such as Type 2 diabetes, and therapeutic methods for treating or preventing the onset of obesity and obesity-related disorders. Methods are also disclosed for identifying a subject, such as a non-obese adolescent, as having a propensity to develop inflammatory obesity.

### CROSS REFERENCE TO RELATED APPLICATIONS

This application claims benefit of U.S. Provisional Application Ser. No. 61/896,446, filed Oct. 28, 2013, the contents of which are incorporated herein by reference.

### BACKGROUND OF THE INVENTION

The prevalence of pediatric obesity has increased worldwide in recent years and raised urgent concern about the on-set of metabolic dysregulation and serious comorbidities, such as Type 2 diabetes, as these obese children reach adulthood. It is likely that adult-onset, obesity-associated pathology is related to diet-induced childhood obesity. Therefore, the identification of pediatric subjects at risk for adult-obesity and related metabolic syndrome could prevent the onset of pediatric obesity and provide effective interventions to reduce risk of Type 2 diabetes and other metabolic diseases. Accordingly, improved methods for identifying subjects, particularly children, at risk for metabolic syndrome and/or obesity are urgently required. Such identification could prevent the weight gain that increases the risk of metabolic disease. The study of obesity, Type 2 diabetes mellitus (T2DM), and related-metabolic diseases in children is complicated by the need for drawing blood from children who fear needles. Therefore, it is desirable to have a non-invasive approach to identify children at risk for metabolic syndrome, Type 2 diabetes, and/or obesity.

### SUMMARY OF THE INVENTION

As described below, the present invention features panels of salivary biomarkers useful for identifying subjects having or at risk of developing a metabolic disease, such as Type 2 diabetes, and therapeutic methods for treating or preventing the onset of obesity and obesity-related disorders.

In one aspect, the invention generally provides a method of characterizing a subject (e.g., child, adolescent) as having or at risk of developing metabolic syndrome, the method involving detecting (e.g., by Western blot, enzyme-linked immunoassay, direct immunoassay, radiometric assay, fluorescence, or protein activity) an alteration in the level of one or more of the following markers C-reactive protein (CRP), insulin, glucose, leptin, and adiponectin in a saliva sample of the subject relative to a reference, thereby characterizing the subject as having or at risk of developing a metabolic syndrome.

In another aspect, the invention provides a method of detecting inflammatory obesity or a propensity to develop inflammatory obesity in a subject, the method involving detecting (e.g., by Western blot, enzyme-linked immunoassay, direct immunoassay, radiometric assay, fluorescence, or protein activity) an alteration in the level of one or more of the following markers CRP, insulin, glucose, IL-6, IL-10, resistin, IL-1beta, MMP-9, and adiponectin in a saliva sample from the subject relative to a reference, thereby detecting inflammatory obesity or a propensity to develop inflammatory obesity in the subject.

In yet another aspect, the invention provides a method of identifying a non-obese subject as having or having a propensity to develop a metabolic syndrome or inflammatory obesity, the method involving detecting (e.g., by Western blot, enzyme-linked immunoassay, direct immunoassay, radiometric assay, fluorescence, or protein activity) an alteration in the level of one or more of the following markers CRP, insulin, glucose, IL-6, IL-10, resistin, IL-1beta, MMP-9, and adiponectin relative to a reference; thereby identifying the subject as having or having a propensity to develop a metabolic syndrome or inflammatory obesity.

In still another aspect, the invention provides a method of identifying a non-obese subject as having a propensity to develop inflammatory obesity involving detecting alterations in the level of markers adiponectin, insulin, glucose, leptin and C-reactive protein (CRP) in a salivary sample of the subject relative to a reference, thereby identifying the subject as having or having a propensity to develop inflammatory obesity.

In still another aspect, the invention provides a method for identifying a subject as in need of therapeutic intervention to prevent or treat a metabolic disorder, the method involving detection of increased levels of C-reactive protein (CRP), glucose, insulin, leptin and reduced levels of adiponectin identify the subject as in need of therapeutic intervention to prevent or treat a metabolic disorder. In one particular embodiment, the therapeutic intervention is any one or more of dietary restriction, increased exercise, or treatment with an anti-inflammatory agent.

In still another aspect, the invention provides a biomarker panel containing C-reactive protein (CRP), insulin, glucose, leptin, and Adiponectin or capture molecules that specifically bind said biomarkers. In one embodiment, the panel further includes one or more of Resistin, IL-8, VEGF, MCP-1, IL-1β, MPO, MMP-9, IL-12P70, IL-4, IL-6, IL-13, TNF-alpha, IL-10, IFN-gamma, Ghrelin, and IL-17A.

In still another aspect, the invention provides a marker panel including CRP, insulin, glucose, IL-6, IL-10, resistin, IL-1beta, MMP-9, and adiponectin or capture molecules that specifically bind those biomarkers.

In various embodiments of the above aspects, or any other aspect of the invention delineated herein the level of adiponectin is decreased relative to the reference (e.g., decreased by at least about 10%, 20%, 30% or more); the level of insulin is increased relative to a reference (e.g., increased by about 2, 3, or 4 times relative to a reference or by at least about 10%, 20%, 30% or more); the level or function of leptin is increased relative to a reference (e.g., increased by about 2, 3. 4 times relative to a reference or by at least about 10%, 20%, 30% or more); the level of C-reactive protein (CRP) is increased relative to a reference (e.g., increased by about 2, 3, 4, 5, 6, or 7 times relative to a reference or by at least about 10%, 20%, 30%, 40%, 50%, 60%, 75% or more); and the increase in salivary glucose level indicates a high plasma glucose level (e.g., a ratio of about 1:13.5 of a plasma glucose level at plasma glucose concentrations greater than about 80 mg/dL or at least about 0.06 mg/dL). In various embodiments of the above aspects, or any other aspect of the invention delineated herein the subject is underweight, normal healthy weight, overweight or obese. In various embodiments of the above aspects, or any other aspect of the invention delineated herein, the method further involves comparing clinical measurements (e.g., increased blood pressure, increased body mass index (BMI), and increased waist circumference) of the subject relative to the reference. In one embodiment, the subject's clinical measurements are increased relative to the reference. In another embodiment, the subject's clinical measurements comprise greater elevation of exercise heart rate relative to the reference. In other embodiments, the subject is a child or adolescent under 18 years of age. In various embodiments of the above aspects, or any other aspect of the invention delineated herein the alteration in polypeptide level is detected by Western blot, enzyme-linked immunoassay, direct immunoassay, radiometric assay, fluorescence, or protein activity. In various embodiments of the above aspects, or any other aspect of the invention delineated herein the reference is a healthy control subject of normal weight, an underweight subject, an overweight subject, or the same subject at an earlier point in time. In particular embodiments of any of the above aspects, the CRP level is greater than about 200-225 pg/ml. In still other embodiments, a non-obese subject is identified as having increased levels of CRP, insulin, glucose, IL-6, IL-10, resistin, IL-1b, and MMP-9. In various embodiments of the above aspects, or any other aspect of the invention delineated herein increased levels of salivary insulin and CRP are indicative of inflammatory obesity or a propensity to develop inflammatory obesity. In still other embodiments, the increased levels of salivary insulin levels, but reduced adiponectin levels are indicative of non-inflammatory obesity. In various embodiments of the above aspects, or any other aspect of the invention delineated herein, the methods further involve measuring a biomarker selected from the group consisting of Adiponectin, Resistin, IL-8, VEGF, MCP-1, CRP, Insulin, glucose, IL-1β, MPO, MMP-9, IL-12P70, IL-4, IL-6, IL-13, TNF-alpha, IL-10, IFN-gamma, Leptin, Ghrelin, and IL-17A.

Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “adiponectin polypeptide” is meant a protein or fragment thereof having at least about 85% amino acid identity to the sequence of GenBank Accession No. NP_001171271, or a fragment thereof, and having at least one adiponectin biological activity. Adiponectin biological activity includes modulating metabolic processes, like glucose regulation and fatty acid oxidation. In one embodiment, an adiponectin polypeptide has at least about 85% amino acid sequence identity to the following amino acid sequence:

| | |
|---|---|
| 1 | mlllgavlll lalpghdqet ttqgpgvllp lpkgactgwm agipghpghn gapgrdgrdg |
| 61 | tpgekgekgd pgligpkgdi getgvpgaeg prgfpgiqgr kgepgegayv yrsafsvgle |
| 121 | tyvtipnmpi rftkifyngq nhydgstgkf hcnipglyyf ayhitvymkd vkvslfkkdk |
| 181 | amlftydqyq ennvdgasgs vllhlevgdq vwlqvygege rnglyadndn dstftgflly |
| 241 | hdtn |

By “adiponectin polynucleotide” is meant a nucleic acid molecule encoding an adiponectin polypeptide or fragment thereof. An exemplary adiponectin nucleic acid sequence (GenBank Accession No. NM_001177800) is provided below:

| | |
|---|---|
| 1 | aggctgttga ggctgggcca tctcctcctc acttccattc tgactgcagt ctgtggttct |
| 61 | gattccatac cagaggagac gggatttcac catgttgtcc aggctggtct gaaactcctg |
| 121 | acatcagggc tcaggatgct gttgctggga gctgttctac tgctattagc tctgcccggt |
| 181 | catgaccagg aaaccacgac tcaagggccc ggagtcctgc ttcccctgcc caagggggcc |
| 241 | tgcacaggtt ggatggcggg catcccaggg catccgggcc ataatggggc cccaggccgt |
| 301 | gatggcagag atggcacccc tggtgagaag ggtgagaaag gagatccagg tcttattggt |
| 361 | cctaagggag acatcggtga aaccggagta cccggggctg aaggtccccg aggctttccg |
| 421 | ggaatccaag gcaggaaagg agaacctgga gaaggtgcct atgtataccg ctcagcattc |
| 481 | agtgtgggat tggagactta cgttactatc cccaacatgc ccattcgctt taccaagatc |
| 541 | ttctacaatc agcaaaacca ctatgatggc tccactggta aattccactg caacattcct |
| 601 | gggctgtact actttgccta ccacatcaca gtctatatga aggatgtgaa ggtcagcctc |
| 661 | ttcaagaagg acaaggctat gctcttcacc tatgatcagt accaggaaaa taatgtggac |
| 721 | caggcctccg gctctgtgct cctgcatctg gaggtgggcg accaagtctg gctccaggtg |
| 781 | tatggggaag gagagcgtaa tggactctat gctgataatg acaatgactc caccttcaca |
| 841 | ggctttcttc tctaccatga caccaactga tcaccactaa ctcagagcct cctccaggcc |
| 901 | aaacagcccc aaagtcaatt aaaggctttc agtacggtta ggaagttgat tattatttag |
| 961 | ttggaggcct ttagatatta ttcattcatt tactcattca tttattcatt cattcatcga |
| 1021 | gtaactttaa aaaaatcata tgctatgttc ccagtcctgg ggagcttcac aaacatgacc |
| 1081 | agataactga ctagaaagaa gtagttgaca gtgctatttt gtgcccactg tctctcctga |
| 1141 | tgctcatatc aatcctataa ggcacaggga acaagcattc tcctgttttt acagattgta |
| 1201 | tcctgaggct gagagagtta agtgaatgtc taaggtcaca cagtattaag tgacagtgct |
| 1261 | agaaatcaaa cccagagctg tggactttgt tcactagact gtgccctttt atagaggtac |
| 1321 | atgttctctt tggagtgttg gtaggtgtct gtttcccacc tcacctgaga gccattgaat |
| 1381 | ttgccttcct catgaattaa aacctccccc aagcagagct tcctcagaga aagtggttct |
| 1441 | atgatgacgt cctgtcttgg aaggactact actcaatggc ccctgcacta ctctacttcc |
| 1501 | tcttacctat gtcccttctc atgcctttcc ctccaacggg gaaagccaac tccatctcta |
| 1561 | agtgccgaac tcatccctgt tcctcaaggc cacctggcca ggagcttctc tgatgtgata |
| 1621 | tccacttttt ttttttttga gatggagtct cactctgtca cccaggctgg agtacagtga |
| 1681 | cacgacctcg gctcactgca gcctccttct cctgggtcca agcaattatt gtgcctcagc |
| 1741 | ctcccgagta gctgagactt caggtgcatt ccaccacaca tggctaattt ttgtattttt |
| 1801 | agtagaaatg gggtttcgtc atgttggcca ggctggtctc gaactcctgg cctaggtgat |
| 1861 | ccacccgcct cgacctccca aagtgctggg attacaggca tgagccacca tgcccagtcg |
| 1921 | atatctcact ttttattttg ccatggatga gagtcctggg tgtgaggaac acctcccacc |
| 1981 | aggctagagg caactgccca ggaaggactg tgcttccgtc acctctaaat cccttgcaga |
| 2041 | tccttgataa atgcctcatg aagaccaatc tcttgaatcc catatctacc cagaattaac |
| 2101 | tccattccag tctctgcatg taatcagttt tatccacaga aacattttca ttttaggaaa |
| 2161 | tccctggttt taagtatcaa tccttgttca gctggacaat atgaatcttt tccactgaag |
| 2221 | ttagggatga ctgtgatttt cagaacacgt ccagaatttt tcatcaagaa ggtagcttga |
| 2281 | gcctgaaatg caaaacccat ggaggaattc tgaagccatt gtctccttga gtaccaacag |
| 2341 | ggtcagggaa gactgggcct cctgaattta ttattgttct ttaagaatta caggttgagg |
| 2401 | tagttgatgg tggtaaacat tctctcagga gacaataact ccagtgatgt tcttcaaaga |
| 2461 | ttttagcaaa aacagagtaa atagcattct ctatcaatat ataaatttaa aaaactatct |
| 2521 | ttttgcttac agttttaaat tctgaacaat tctctcttat atgtgtattg ctaatcatta |
| 2581 | aggtattatt ttttccacat ataaagcttt gtctttttgt tgttgttgtt gtttttaaga |
| 2641 | tggagtttcc ctctgttgcc aggctagagt gcagtggcat gatctcggct tactgcaacc |
| 2701 | tttgcctccc aggttcaagc gattcttctg cctcagcctc ccgagtagct gggaccacag |
| 2761 | gtgcctacca ccatgccagg ctaatttttg tatttttagt aaagacaggg tttcaccata |
| 2821 | ttggccaggc tggtctcgaa ctcctgacct tgtgatctgc ccgcctccat ttttgttgtt |
| 2881 | attttttgag aaagatagat atgaggttta gagagggatg aagaggtgag agtaagcctt |
| 2941 | gtgttagtca gaactctgtg ttgtgaatgt cattcacaac agaaaaccca aaatattatg |
| 3001 | caaactactg taagcaagaa aaataaagga aaaatggaaa catttattcc tttgcataat |
| 3061 | agaaattacc agagttgttc tgtctttaga taaggtttga accaaagctc aaaacaatca |
| 3121 | agaccctttt ctgtatgtcc ttctgttctg ccttccgcag tgtaggcttt accctcaggt |
| 3181 | gctacacagt atagttctag ggtttccctc ccgatatcaa aaagactgtg gcctgcccag |
| 3241 | ctctcgtatc cccaagccac accatctggc taaatggaca tcatgttttc tggtgatgcc |
| 3301 | caaagaggag agaggaagct ctctttccca gatgccccag caagtgtaac cttgcatctc |
| 3361 | attgctctgg ctgagttgtg tgcctgtttc tgaccaatca ctgagtcagg aggatgaaat |
| 3421 | attcatattg acttaattgc agcttaagtt aggggtatgt agaggtattt tccctaaagc |
| 3481 | aaaattggga cactgttatc agaaatagga gagtggatga tagatgcaaa ataatacctg |
| 3541 | tccacaacaa actcttaatg ctgtgtttga gctttcatga gtttcccaga gagacatagc |
| 3601 | tggaaaattc ctattgattt tctctaaaat ttcaacaagt agctaaagtc tggctatgct |
| 3661 | cacagtctca catctggttg gggtgggctc cttacagaac acgctttcac agttacccta |
| 3721 | aactctctgg ggcagggtta ttcctttgtg gaaccagagg cacagagaga gtcaactgag |
| 3781 | gccaaaagag gcctgagaga aactgaggtc aagatttcag gattaatggt cctgtgatgc |
| 3841 | tttgaagtac aattgtggat ttgtccaatt ctctttagtt ctgtcagctt ttgcttcata |
| 3901 | tattttagcg ctctattatt agatatatac atgtttagta ttatgtctta ttggtgcatt |
| 3961 | tactctctta tcattatgta atgtccttct ttatctgtga taattttctg tgttctgaag |
| 4021 | tctactttgt ctaaaaataa catacgcact caacttcctt ttctttcttc cttcctttct |
| 4081 | ttcttccttc ctttctttct ctctctctct ctttccttcc ttccttcctc cttttctttc |
| 4141 | tctctctctc tctctctctt tttttgacag actctcgttc tgtggccctg gctggagttc |
| 4201 | agtggtgtga tcttggctca ctgctacctc taccatgagc aattctcctg cctcagcctc |
| 4261 | ccaagtagct ggaactacag gctcatgcca ctgcgcccag ctaatttttg tatttttcgt |
| 4321 | agagacgggg tttcaccaca ttcgtcaggt tggtttcaaa ctcctgactt tgtgatccac |
| 4381 | ccgcctcggc ctcccaaagt gctgggatta caggcatgag ccatcacacc tggtcaactt |
| 4441 | tcttttgatt agtgtttttg tggtatatct ttttccatca tgttacttta aatatatcta |
| 4501 | tattattgta tttaaaatgt gtttcttaca gactgcatgt agttgggtat aatttttatc |
| 4561 | cagtctaaaa atatctgtct tttaattggt gtttagacaa tttatattta ataaaattgt |
| 4621 | tgaatttaa |

By “C-reactive protein (CRP) polypeptide” is meant a protein having at least about 85% amino acid identity to the sequence of GenBank Accession No. NP_000558, or a fragment thereof, and having at least one CRP biological activity. CRP biological activity includes binding phosphocholines to activate the complement system. In one embodiment, a CRP polypeptide has at least about 85% amino acid sequence identity to the following amino acid sequence:

| | |
|---|---|
| 1 | mekllcflvl tslshafgqt dmsrkafvfp kesdtsyvsl kapltkplka ftvclhfyte |
| 61 | lsstrgysif syatkrqdne ilifwskdig ysftvggsei lfevpevtva pvhictswes |
| 121 | asgivefwvd gkprvrkslk kgytvgaeas iilggegdsf ggnfegsgsl vgdignvnmw |
| 181 | dfvlspdein tiylggpfsp nvinwralky evqgevftkp qlwp |

By “CRP polynucleotide” is meant a nucleic acid molecule encoding a CRP polypeptide or fragment thereof. An exemplary CRP nucleic acid sequence (GenBank Accession No. NM_000567) is provided below:

| | |
|---|---|
| 1 | aaggcaagag atctaggact tctagcccct gaactttcag ccgaatacat cttttccaaa |
| 61 | ggagtgaatt caggcccttg tatcactggc agcaggacgt gaccatggag aagctgttgt |
| 121 | gtttcttggt cttgaccagc ctctctcatg cttttggcca gacagacatg tcgaggaagg |
| 181 | cttttgtgtt tcccaaagag tcggatactt cctatgtatc cctcaaagca ccgttaacga |
| 241 | agcctctcaa agccttcact gtgtgcctcc acttctacac ggaactgtcc tcgacccgtg |
| 301 | ggtacagtat tttctcgtat gccaccaaga gacaagacaa tgagattctc atattttggt |
| 361 | ctaaggatat aggatacagt tttacagtgg gtgggtctga aatattattc gaggttcctg |
| 421 | aagtcacagt agctccagta cacatttgta caagctggga gtccgcctca gggatcgtgg |
| 481 | agttctgggt agatgggaag cccagggtga ggaagagtct gaagaaggga tacactgtgg |
| 541 | gggcagaagc aagcatcatc ttggggcagg agcaggattc cttcggtggg aactttgaag |
| 601 | gaagccagtc cctggtggga gacattggaa atgtgaacat gtgggacttt gtgctgtcac |
| 661 | cagatgagat taacaccatc tatcttggcg ggcccttcag tcctaatgtc ctgaactggc |
| 721 | gggcactgaa gtatgaagtg caaggcgaag tgttcaccaa accccagctg tggccctgag |
| 781 | gcccagctgt gggtcctgaa ggtacctccc ggttttttac accgcatggg ccccacgtct |
| 841 | ctgtctctgg tacctcccgc ttttttacac tgcatggttc ccacgtctct gtctctgggc |
| 901 | ctttgttccc ctatatgcat tgcaggcctg ctccaccctc ctcagcgcct gagaatggag |
| 961 | gtaaagtgtc tggtctggga gctcgttaac tatgctggga aacggtccaa aagaatcaga |
| 1021 | atttgaggtg ttttgttttc atttttattt caagttggac agatcttgga gataatttct |
| 1081 | tacctcacat agatgagaaa actaacaccc agaaaggaga aatgatgtta taaaaaactc |
| 1141 | ataaggcaag agctgagaag gaagcgctga tcttctattt aattccccac ccatgacccc |
| 1201 | cagaaagcag gagggcattg cccacattca cagggctctt cagtctcaga atcaggacac |
| 1261 | tggccaggtg tctggtttgg gtccagagtg ctcatcatca tgtcatagaa ctgctgggcc |
| 1321 | caggtctcct gaaatgggaa gcccagcaat accacgcagt ccctccactt tctcaaagca |
| 1381 | cactggaaag gccattagaa ttgccccagc agagcagatc tgcttttttt ccagagcaaa |
| 1441 | atgaagcact aggtataaat atgttgttac tgccaagaac ttaaatgact ggtttttgtt |
| 1501 | tgcttgcagt gctttcttaa ttttatggct cttctgggaa actcctcccc ttttccacac |
| 1561 | gaaccttgtg gggctgtgaa ttctttcttc atccccgcat tcccaatata cccaggccac |
| 1621 | aagagtggac gtgaaccaca gggtgtcctg tcagaggagc ccatctccca tctccccagc |
| 1681 | tccctatctg gaggatagtt ggatagttac gtgttcctag caggaccaac tacagtcttc |
| 1741 | ccaaggattg agttatggac tttgggagtg agacatcttc ttgctgctgg atttccaagc |
| 1801 | tgagaggacg tgaacctggg accaccagta gccatcttgt ttgccacatg gagagagact |
| 1861 | gtgaggacag aagccaaact ggaagtggag gagccaaggg attgacaaac aacagagcct |
| 1921 | tgaccacgtg gagtctctga atcagccttg tctggaacca gatctacacc tggactgccc |
| 1981 | aggtctataa gccaataaag cccctgttta cttgaaaaaa aaaa |

By “insulin polypeptide” is meant a protein having at least about 85% amino acid identity to the sequence of GenBank Accession No. NP_000198, or a fragment thereof, and having at least one insulin biological activity. Insulin biological activity includes regulating carbohydrate and fat metabolism in the body and signaling cells to absorb glucose from the blood. In one embodiment, an insulin polypeptide has at least about 85% amino acid sequence identity to the following amino acid sequence:

| | |
|---|---|
| 1 | malwmrllpl lallalwgpd paaafvnqhl cgshlvealy lvcgergffy tpktrreaed |
| 61 | lqvggvelgg gpgagslqpl alegslqkrg iveqcctsic slyglenycn |

By “insulin polynucleotide” is meant a nucleic acid molecule encoding an insulin polypeptide or fragment thereof. An exemplary insulin nucleic acid sequence (GenBank Accession No. NM_000207) is provided below:

| | |
|---|---|
| 1 | agccctccag gacaggctgc atcagaagag gccatcaagc agatcactgt ccttctgcca |
| 61 | tggccctgtg gatgcgcctc ctgcccctgc tggcgctgct ggccctctgg ggacctgacc |
| 121 | cagccgcagc ctttgtgaac caacacctgt gcggctcaca cctggtggaa gctctctacc |
| 181 | tagtgtgcgg ggaacgaggc ttcttctaca cacccaagac ccgccgggag gcagaggacc |
| 241 | tgcaggtggg gcaggtggag ctgggcgggg gccctggtgc aggcagcctg cagcccttgg |
| 301 | ccctggaggg gtccctgcag aagcgtggca ttgtggaaca atgctgtacc agcatctgct |
| 361 | ccctctacca gctggagaac tactgcaact agacgcagcc cgcaggcagc cccacacccg |
| 421 | ccgcctcctg caccgagaga gatggaataa agcccttgaa ccagcaaaa |

By “leptin polypeptide” is meant a protein having at least about 85% amino acid identity to the sequence of GenBank Accession No. NP_000221, or a fragment thereof, and having at least one leptin biological activity. Leptin biological activity includes counteracting the effects of neuropeptide Y and anandamide and promoting appetite suppression. In one embodiment, a leptin polypeptide has at least about 85% amino acid sequence identity to the following amino acid sequence:

| | |
|---|---|
| 1 | mhwgticgfl wlwpylfyvq avpiqkvqdd tktliktivt rindishtqs vsskqkvtgl |
| 61 | dfipglhpil tlskmdqtla vyqqiltsmp srnvigisnd lenlrdllhv lafskschlp |
| 121 | wasgletlds lggvleasgy stevvalsrl qgslqdmlwq ldlspgc |

By “leptin polynucleotide” is meant a nucleic acid molecule encoding a leptin polypeptide or fragment thereof. An exemplary leptin nucleic acid sequence (GenBank Accession No. NM_000230) is provided below:

| | |
|---|---|
| 1 | gtaggaatcg cagcgccagc ggttgcaagg cccaagaagc ccatcctggg aaggaaaatg |
| 61 | cattggggaa ccctgtgcgg attcttgtgg ctttggccct atcttttcta tgtccaagct |
| 121 | gtgcccatcc aaaaagtcca agatgacacc aaaaccctca tcaagacaat tgtcaccagg |
| 181 | atcaatgaca tttcacacac gcagtcagtc tcctccaaac agaaagtcac cggtttggac |
| 241 | ttcattcctg ggctccaccc catcctgacc ttatccaaga tggaccagac actggcagtc |
| 301 | taccaacaga tcctcaccag tatgccttcc agaaacgtga tccaaatatc caacgacctg |
| 361 | gagaacctcc gggatcttct tcacgtgctg gccttctcta agagctgcca cttgccctgg |
| 421 | gccagtggcc tggagacctt ggacagcctg gggggtgtcc tggaagcttc aggctactcc |
| 481 | acagaggtgg tggccctgag caggctgcag gggtctctgc aggacatgct gtggcagctg |
| 541 | gacctcagcc ctgggtgctg aggccttgaa ggtcactctt cctgcaagga ctacgttaag |
| 601 | ggaaggaact ctggcttcca ggtatctcca ggattgaaga gcattgcatg gacacccctt |
| 661 | atccaggact ctgtcaattt ccctgactcc tctaagccac tcttccaaag gcataagacc |
| 721 | ctaagcctcc ttttgcttga aaccaaagat atatacacag gatcctattc tcaccaggaa |
| 781 | gggggtccac ccagcaaaga gtgggctgca tctgggattc ccaccaaggt cttcagccat |
| 841 | caacaagagt tgtcttgtcc cctcttgacc catctccccc tcactgaatg cctcaatgtg |
| 901 | accaggggtg atttcagaga gggcagaggg gtaggcagag cctttggatg accagaacaa |
| 961 | ggttccctct gagaattcca aggagttcca tgaagaccac atccacacac gcaggaactc |
| 1021 | ccagcaacac aagctggaag cacatgttta tttattctgc attttattct ggatggattt |
| 1081 | gaagcaaagc accagcttct ccaggctctt tggggtcagc cagggccagg ggtctccctg |
| 1141 | gagtgcagtt tccaatccca tagatgggtc tggctgagct gaacccattt tgagtgactc |
| 1201 | gagggttggg ttcatctgag caagagctgg caaaggtggc tctccagtta gttctctcgt |
| 1261 | aactggtttc atttctactg tgactgatgt tacatcacag tgtttgcaat ggtgttgccc |
| 1321 | tgagtggatc tccaaggacc aggttatttt aaaaagattt gttttgtcaa gtgtcatatg |
| 1381 | taggtgtctg cacccagggg tggggaatgt ttgggcagaa gggagaagga tctagaatgt |
| 1441 | gttttctgaa taacatttgt gtggtgggtt ctttggaagg agtgagatca ttttcttatc |
| 1501 | ttctgcaatt gcttaggatg tttttcatga aaatagctct ttcagggggg ttgtgaggcc |
| 1561 | tggccaggca ccccctggag agaagtttct ggccctggct gaccccaaag agcctggaga |
| 1621 | agctgatgct ttgcttcaaa tccatccaga ataaaacgca aagggctgaa agccatttgt |
| 1681 | tggggcagtg gtaagctctg gctttctccg actgctaggg agtggtcttt cctatcatgg |
| 1741 | agtgacggtc ccacactggt gactgcgatc ttcagagcag gggtccttgg tgtgaccctc |
| 1801 | tgaatggtcc agggttgatc acactctggg tttattacat ggcagtgttc ctatttgggg |
| 1861 | cttgcatgcc aaattgtagt tcttgtctga ttggctcacc caagcaaggc caaaattacc |
| 1921 | aaaaatcttg gggggttttt actccagtgg tgaagaaaac tcctttagca ggtggtcctg |
| 1981 | agacctgaca agcactgcta ggcgagtgcc aggactcccc aggccaggcc accaggatgg |
| 2041 | cccttcccac tggaggtcac attcaggaag atgaaagagg aggtttgggg tctgccacca |
| 2101 | tcctgctgct gtgtttttgc tatcacacag tgggtggtgg atctgtccaa ggaaacttga |
| 2161 | atcaaagcag ttaactttaa gactgagcac ctgcttcatg ctcagccctg actggtgcta |
| 2221 | taggctggag aagctcaccc aataaacatt aagattgagg cctgccctca gggatcttgc |
| 2281 | attcccagtg gtcaaaccgc actcacccat gtgccaaggt ggggtattta ccacagcagc |
| 2341 | tgaacagcca aatgcatggt gcagttgaca gcaggtggga aatggtatga gctgaggggg |
| 2401 | gccgtgccca ggggcccaca gggaaccctg cttgcacttt gtaacatgtt tacttttcag |
| 2461 | ggcatcttag cttctattat agccacatcc ctttgaaaca agataactga gaatttaaaa |
| 2521 | ataagaaaat acataagacc ataacagcca acaggtggca ggaccaggac tatagcccag |
| 2581 | gtcctctgat acccagagca ttacgtgagc caggtaatga gggactggaa ccagggagac |
| 2641 | cgagcgcttt ctggaaaaga ggagtttcga ggtagagttt gaaggaggtg agggatgtga |
| 2701 | attgcctgca gagagaagcc tgttttgttg gaaggtttgg tgtgtggaga tgcagaggta |
| 2761 | aaagtgtgag cagtgagtta cagcgagagg cagagaaaga agagacagga gggcaagggc |
| 2821 | catgctgaag ggaccttgaa gggtaaagaa gtttgatatt aaaggagtta agagtagcaa |
| 2881 | gttctagaga agaggctggt gctgtggcca gggtgagagc tgctctggaa aatgtgaccc |
| 2941 | agatcctcac aaccacctaa tcaggctgag gtgtcttaag ccttttgctc acaaaacctg |
| 3001 | gcacaatggc taattcccag agtgtgaaac ttcctaagta taaatggttg tctgtttttg |
| 3061 | taacttaaaa aaaaaaaaaa aagtttggcc gggtgcggtg gctcacgcct gtaatcccag |
| 3121 | cactttggga ggccaaggtg gggggatcac aaggtcacta gatggcgagc atcctggcca |
| 3181 | acatggtgaa accccgtctc tactaaaaac acaaaagtta gctgagcgtg gtggcgggcg |
| 3241 | cctgtagtcc cagccactcg ggaggctgag acaggagaat cgcttaaacc tgggaggcgg |
| 3301 | agagtacagt gagccaagat cgcgccactg cactccggcc tgatgacaga gcgagattcc |
| 3361 | gtcttaaaaa aaaaaaaaaa aaagtttgtt tttaaaaaaa tctaaataaa ataactttgc |
| 3421 | cccctgcaaa aaaaaaaaaa aaaa |

By “metabolic syndrome” is meant one or more conditions associated with an increased risk of cardiovascular disease, stroke and/or diabetes. Metabolic syndrome includes conditions associated with increased blood pressure, high blood sugar level, excess body fat around the waist and abnormal cholesterol levels.

As used herein, the term “obesity” refers to a body mass index (BMI) of 30 kg/m² or more (National Institute of Health, Clinical Guidelines on the Identification, Evaluation, and Treatment of Overweight and Obesity in Adults (1998)). However, the present invention is also intended to include a disease, disorder, or condition that is characterized by a body mass index (BMI) of 25 kg/m² or more, 26 kg/m² or more, 27 kg/m² or more, 28 kg/m² or more, 29 kg/m² or more, 29.5 kg/m² or more, or 29.9 kg/m² or more, all of which are typically referred to as overweight (National Institute of Health, Clinical Guidelines on the Identification, Evaluation, and Treatment of Overweight and Obesity in Adults (1998)).

By “alteration” is meant a change (increase or decrease) in the expression levels or activity of a marker or clinical indicator as detected by standard art known methods such as those described herein. As used herein, an alteration includes a 10%-100% change in measured levels (e.g., 10, 20, 30, 40, 50, 60, 75, 80, 85, 90, 95, 100%).

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

As used herein, the term “diabetes” includes both insulin-dependent diabetes mellitus (i.e., IDDM, also known as Type 1 diabetes) and non-insulin-dependent diabetes mellitus (i.e., NIDDM, also known as Type 2 diabetes). Type 1 diabetes, or insulin-dependent diabetes, is the result of an absolute deficiency of insulin, the hormone which regulates glucose utilization. Type 2 diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type 2 diabetics are also obese. The World Health Organization defines the diagnostic value of fasting plasma glucose concentration to 7.0 mmol/l (126 mg/dl) and above for Diabetes Mellitus (whole blood 6.1 mmol/l or 110 mg/dl), or 2-hour glucose level of 11.1 mmol/L or more (200 mg/dL or more). Other values suggestive of or indicating high risk for diabetes mellitus include elevated arterial pressure of 140/90 mm Hg or more; elevated plasma triglycerides (1.7 mmol/L or 150 mg/dL or more) and/or low HDL-cholesterol (<0.9 mmol/L, 35 mg/dl for men; <1.0 mmol/L, 39 mg/dL women); central obesity (males: waist to hip ratio >0.90; females: waist to hip ratio >0.85) and/or body mass index exceeding 30 kg/m²; microalbuminuria, where the urinary albumin excretion rate is 20 ug/min or albumin:creatinine ratio 30 mg/g). Type 1 Diabetes can also be distinguished from Type 2 diabetes using a C-peptide assay, which is a measure of endogenous insulin production. The presence of anti-islet antibodies (to Glutamic Acid Decarboxylase, Insulinoma Associated Peptide-2 or insulin), or lack of insulin resistance, determined by a glucose tolerance test, is also indicative of Type 1, as many Type 2 diabetics continue to produce insulin internally, and all have some degree of insulin resistance.

“Diagnostic” means identifying the presence or nature of a pathologic condition. While a particular diagnostic method may not provide a definitive diagnosis of a condition, it suffices if the method provides a positive indication that aids in diagnosis.

The phrase “differentially present” refers to differences in the quantity and/or the frequency of a marker present in a sample taken from subjects having a disease as compared to a control subject. A marker can be differentially present in terms of quantity, frequency or both. A polypeptide is differentially present between two samples if the amount of the polypeptide in one sample is statistically significantly different from the amount of the polypeptide in the other sample. Alternatively or additionally, a polypeptide is differentially present between two sets of samples if the frequency of detecting the polypeptide in a diseased subjects' samples is statistically significantly higher or lower than in the control samples. A marker that is present in one sample, but undetectable in another sample is differentially present.

By “disease” is meant any condition or disorder that damages or interferes with the normal function of a cell, tissue, or organ. Examples of diseases include a metabolic syndrome or metabolic-syndrome related diseases, including but not limited to obesity, diabetes, including Type 1 and Type 2 diabetes, insulin-deficiency, insulin-resistance, insulin-resistance related disorders, glucose intolerance, hypoglycemia, syndrome X, inflammatory and immune disorders, dyslipidemia, metabolic syndrome, non-alcoholic fatty liver, abnormal lipid metabolism, sleep apnea, hypertension, high cholesterol, atherogenic dyslipidemia, hyperlipidemic conditions such as atherosclerosis, hypercholesterolemia, and other coronary artery diseases in mammals, and other disorders of metabolism.

By “effective amount” is meant the amount of a compound described herein required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in level or activity that is associated with a disease or disorder.

“Monitoring” refers to recording changes in a continuously varying parameter (e.g. monitoring progression of a disease).

As used herein, “obtaining” as in “obtaining an agent” includes synthesizing, purchasing, or otherwise acquiring the agent.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reduces” is meant a negative alteration of at least 10%, 25%, 50%, 75%, or 100%.

By “reference” is meant a standard or control condition.

As used herein, “sample” or “biological sample” refers to anything, which may contain an analyte (e.g., polypeptide, polynucleotide, or fragment thereof) for which an analyte assay is desired. The sample may be a biological sample, such as a biological fluid or a biological tissue. In one embodiment, a biological sample is a salivary sample. Such a sample may include diverse cells, proteins, and genetic material. Examples of biological tissues also include organs, tumors, lymph nodes, arteries and individual cell(s). Examples of biological fluids include urine, blood, plasma, serum, saliva, semen, stool, sputum, cerebral spinal fluid, tears, mucus, amniotic fluid or the like.

As used herein, the term “sensitivity” is the percentage of marker-detected subjects with a particular disease.

By “specifically binds” is meant a reagent that recognizes and binds a polypeptide of the invention, but which does not substantially recognize and bind other molecules in a sample, for example, a biological sample, which naturally includes a polypeptide of the invention.

By “subject” is meant a mammal, including, but not limited to, a human or non-human mammal, such as a bovine, equine, canine, ovine, or feline.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Any compositions or methods provided herein can be combined with one or more of any of the other compositions and methods provided herein.

### BRIEF DESCRIPTION OF THE DRAWINGS

FIG. 1 is a plot of insulin concentrations in fasting saliva and plasma of 53 adolescent donors. These data were fitted after log transformation to obtain the equation Ln(Plasma)=0.85*Ln(Saliva)+1.84 with r²=0.67. Based on this approximation, the predictor variable of 128 pg/ml saliva insulin would be approximately 68 pmoles/L of plasma insulin (or 11 μU/ml using the conversion factor 1 μU/ml=6.00 pmol/L⁵³).

FIGS. 2A-2D provide four graphs of salivary biomarkers that were significantly different in obese children compared to normal weight children. Central values represent the median concentration and whiskers represent the interquartile range (+75^{th} percentile, −25^{th} percentile). FIG. 2A is a graph showing insulin is significantly increased with obesity. FIG. 2B is a graph showing C-reactive protein (CRP) is significantly increased with obesity. FIG. 2C is a graph showing leptin is significantly increased with obesity. FIG. 2D is a graph showing adiponectin is significantly decreased with obesity;

FIG. 3 is a bar graph of salivary concentrations of biomarkers tested by body weight category. Values represent medians (center bar) +25^{th} percentile and −75^{th} percentile on a logarithmic axis for each category; and

FIG. 4 is a categorical decision tree describing identification of children that are obese or non-obese. 76% of the obese children were identified by the predictor variable salivary CRP>219 pg/ml. Of the lower salivary CRP saliva samples, 13% of the obese children were identified by the predictor variable insulin >128 pg/ml and 11% had insulin ≦128 pg/ml.

FIG. 5 is a scatter graph showing saliva glucose concentration (Sg) as a function of salivary flow rate (F) by regression analysis; and

FIGS. 6A-C provide two graphs and an analysis of plasma glucose concentration as a function of salivary glucose concentration. FIG. 6A is a scatter graph of a regression analysis of samples from all of the children in the study (mean age 10.6±0.2 y). FIG. 6B shows a 2×2 analysis of the diagnostic capability of salivary glucose testing to identify children with high plasma glucose levels (>90 mg/dL) using a value of 0.06 mg/dL in saliva as an identification criterion. TP=true positive (red), FP=false positive (green), TN=true negative (black), FN=false negative (blue), PPV=positive predictive value, NPV=negative predictive value. FIG. 6C is a graph depicting a receiver operating curve indicating an area under the curve (AUC) measurement of 0.78.

### DETAILED DESCRIPTION OF THE INVENTION

The invention features panels of salivary biomarkers useful for identifying subjects having or at risk of developing a metabolic disease, such as Type 2 diabetes, and therapeutic methods for treating or preventing the onset of obesity and obesity-related disorders.

The invention is based, at least in part, on the discovery that metabolic and inflammatory biomarkers are present in altered levels in the saliva of subjects having or at risk of developing a metabolic syndrome, obesity, or diabetes. As reported in greater detail herein below, twenty salivary biomarkers: Adiponectin, Resistin, IL-8, VEGF, MCP-1, CRP, Insulin, IL-10, MPO, MMP-9, IL-12P70, IL-4, IL-6, IL-13, TNF-alpha, IL-10, IFN-gamma, Leptin, Ghrelin, and IL-17A were used to evaluate metabolic changes associated with obesity in 11-year old children (n=744) in four body weight categories; underweight, normal healthy weight, overweight and obese. Salivary C-reactive protein (CRP) was almost 6 times higher and salivary insulin and leptin were approximately 3 times higher (all p <0.0001) in obese children compared to healthy normal weight children. Adiponectin was approximately 30% lower in obese children (p<0.0001) than in healthy normal weight children. This biomarker analysis defined three types of obesity in children: inflammatory obesity, non-inflammatory obesity characterized by high salivary insulin levels, but low levels of inflammatory mediators, and non inflammatory obesity characterized by slightly elevated salivary insulin, but significantly reduced adiponectin levels. Surprisingly, this analysis also identified a group of normal weight children having increased levels of inflammatory biomarkers that likely identify them as at risk for obesity and obesity-related metabolic syndrome.

Seventy-six percent of obese children had high (>219 pg/ml) CRP, with a decidedly inflammatory character. In the children with CRP≦128 pg/ml, 13% of the obese children had high salivary insulin, but no elevated inflammatory mediators and the remaining 11% obese children had only slightly elevated salivary insulin but significantly reduced adiponectin. In addition, 40% of the non-obese children based on biomarker characteristics, identify them as at risk for becoming obese.

Salivary analysis for fasting glucose may be used as a surrogate measurement for the level in plasma. This approach has been tested in adult patients with Type 2 diabetes. Several studies have reported a possible correlation between fasting blood glucose and fasting salivary glucose levels for adult patients exhibiting symptoms of Type 2 diabetes. To date, no studies have been performed evaluating the utility of salivary glucose levels as a surrogate marker for plasma glucose levels in children and adolescents at risk for metabolic syndrome. Therefore, the utility of salivary glucose analysis as a possible screening method for fasting hyperglycemia in children was evaluated herein by comparing the glucose levels in salivary and blood samples given by 11-year-old US children who were either normal weight, overweight, or obese. The results identified panels of salivary biomarkers useful for identifying subjects having or at risk of developing a metabolic disease, such as Type 2 diabetes, and therapeutic methods for treating or preventing the onset of obesity and obesity-related disorders.

### Childhood and Adolescent Obesity

Obesity is a problem whose best solution lies in prevention which in turn, depends on diagnosis. Often diagnosis is avoided because of the invasive nature of blood sampling. As reported herein below, the present invention identifies biomarkers present in saliva useful in characterizing or diagnosing inflammatory obesity and related metabolic syndrome. As pediatric obesity has increased, public health authorities expect to see corresponding increases in metabolic dysregulation and serious comorbidities, such as Type 2 diabetes, as these obese children age into adulthood. The relationship between adult-onset, obesity-associated pathology and exposure of these individuals to diet-induced obesity, while still children, is unknown. Nevertheless, it seems likely that childhood obesity will only compound the serious health consequences associated with adult-onset obesity. If at-risk cohorts of pediatric subjects can be identified using non-invasive measures provided herein, it is possible that the course of pediatric obesity could be reversed, and effective interventions employed to reduce the risk of Type 2 diabetes. The importance of such diagnostic and therapeutic methods is widely recognized considering that countries with significant prevalence of pediatric obesity are devoting significant resources to understanding the scope of this problem and devising appropriate public health recommendations.

### Salivary Biomarkers

Saliva is a readily accessible body fluid that can be obtained in a non-invasive manner Saliva can be used to monitor metabolic and inflammatory biomarkers for the diagnosis of a metabolic syndrome, inflammatory obesity, or a propensity to develop a metabolic syndrome, obesity, or diabetes. Biomarkers present in saliva can also be used to monitor disease progression. Salivary biomarkers useful in the methods of the invention include the adipokines (adipocytokines). Of particular interest were a subset of proteins, including adiponectin, leptin and resistin, which may be described as adipose-derived hormones, that also appear in saliva. Other adipokines include salivary monocyte chemotatic protein-1 (MCP-1) and salivary tumor necrosis factor-alpha (TNF-α). A second major class of investigated biomarkers were the inflammation-cytokines, which include interleukins (IL).

Salivary IL-1β has been associated with periodontal inflammation. Salivary IL-6 has been measured in periodontitis patients. Salivary IL-4, IL-10, IL-12 and IL-17 have been related to Sjogren's syndrome. Salivary IL-10 was also found reduced in periodontitis patients. Salivary IL-8 has been related to dental caries in adolescents. IL-13 has been identified in the sputum of asthmatics. Salivary levels of IL-17 were reported to be lower in patients with periodontal disease. Interferon γ (IFN-γ) was higher in the saliva of control subjects without Sjogren's syndrome. Thus, adipokines and cytokines, from sources other than peripheral blood, have been assayed and associated with disease states.

Both salivary immunoreactive insulin and salivary ghrelin have been associated with Type 2 diabetes and obesity and were included in the analysis. Two oral disease biomarkers were also included, matrix metallopeptidase 9 (MMP-9) and myeloperoxidase (MPO). MMP-9 is a protease often found elevated in patients with periodontal disease and oral cancer. MPO, a peroxidase found abundantly in neutrophil granulocytes and often used as a measure of neutrophil degranulation, has been reported to be elevated in diabetic patients. Vascular endothelial growth factor (VEGF) was also included in the study because of evidence that it has been found in saliva and was elevated in diabetic women. C-reactive protein (CRP) was included because has been found in saliva and has been associated with inflammation and cardiovascular disease.

Collectively, the panel represents various arms of the inflammatory and metabolic processes for testing in saliva using a multiplex method of detection. This provides an important advance given that at-risk populations of children are particularly difficult to diagnosis and monitor disease because of a fear of needles. In one embodiment, a panel of the invention includes but is not limited to any one or more of Adiponectin, Resistin, IL-8, VEGF, MCP-1, CRP, Insulin, IL-1β, MPO, MMP-9, IL-12P70, IL-4, IL-6, IL-13, TNF-alpha, IL-10, IFN-gamma, Leptin, Ghrelin, and IL-17A.

In other embodiments, the invention provides a panel of metabolic and inflammatory biomarkers present in saliva including but not limited to adiponectin, insulin, leptin, and C-reactive protein (CRP). Adiponectin is a protein hormone that modulates a number of metabolic processes, including glucose regulation and fatty acid oxidation. Insulin is a peptide hormone, produced by beta cells of the pancreas, and is central to regulating carbohydrate and fat metabolism in the body. Leptin is a hormone made by fat tissue that acts on brain to regulate food intake and body weight. Inflammatory mechanisms in obesity and defined as low-grade, chronic inflammation orchestrated by metabolic cells in response to excess nutrients and energy, may contribute to metabolic dysfunction, including increases in circulating cytokines. C-reactive protein (CRP) is a protein which rises in response to inflammation. To test whether levels of one or more of these biomarkers in saliva are indicative of metabolic syndrome. Metabolic and inflammatory marker (e.g. adiponectin, insulin, leptin, and CRP) levels were compared in underweight, normal healthy weight, overweight and obese adolescents. These biomarkers can be used alone, or in combination with any one or all of the other biomarkers delineated herein (e.g., Resistin, IL-8, VEGF, MCP-1, IL-1β, MPO, MMP-9, IL-12P70, IL-4, IL-6, IL-13, TNF-alpha, IL-10, IFN-gamma, Ghrelin, and IL-17A).

### Metabolic Syndrome

Markers of the invention are used for the identification of subjects as having or having a propensity to develop metabolic syndrome. Metabolic syndrome is a set of conditions that increase a subjects' risk of heart disease, stroke, and diabetes. Such conditions include obesity, insulin-deficiency, insulin-resistance, insulin-resistance related disorders, glucose intolerance, syndrome X, inflammatory and immune disorders, dyslipidemia, metabolic syndrome, non-alcoholic fatty liver, abnormal lipid metabolism, sleep apnea, hypertension, high cholesterol, atherogenic dyslipidemia, hyperlipidemic conditions, such as atherosclerosis, hypercholesterolemia, and other coronary artery diseases in mammals, and other disease associated with obesity or dysregulated metabolism. In particular embodiments, the invention provides for the use of one or more of altered levels of adiponectin, insulin, leptin and CRP in saliva to evaluate a subject as having or at risk of developing metabolic syndrome, diabetes, or obesity (e.g., inflammatory obesity).

Methods of the invention involve detecting an increase or decrease in the level of a biomarker in a biological sample obtained from a subject (e.g., saliva) relative to the level present in a control. In one embodiment, an increase in the level of insulin, leptin, or CRP relative to a reference is indicative of a metabolic syndrome, obesity, diabetes, or the propensity to develop such a condition. In another embodiment, a decrease in the level of adiponectin relative to a reference is indicative of a metabolic syndrome, obesity, diabetes, or the propensity to develop such a condition.

### Clinical Indicators

The present invention provides metabolic and inflammatory biomarkers whose expression or level is altered in a biological sample derived from a subject having or having a propensity to develop metabolic syndrome, obesity (e.g., inflammatory obesity) or diabetes, or a propensity to develop such conditions. Such biomarkers, which include metabolic and inflammatory biomarkers, may be used individually or in combination with clinical biomarkers or measurements, such as blood pressure, body mass index (BMI), waist circumference, and heart rate, to provide a method of diagnosing and/or monitoring a metabolic syndrome, obesity (e.g., inflammatory obesity) or diabetes, or a propensity to develop such conditions. In some embodiments, the clinical measurements of the subject are compared to the measurements present in a reference (e.g., a standard or control from a healthy control subject, an underweight control subject). In particular embodiments, the subject's clinical measurements, such as blood pressure, BMI, and waist circumference are increased relative to measurements obtained from a reference. The subject's clinical measurements can also include greater elevation of exercise heart rate relative to the reference (e.g., relative to exercise heart rate in a healthy control subject of normal weight).

### Diagnostics

Saliva obtained from obese subjects has altered levels of particular biomarkers. In particular, subjects are identified as having a metabolic syndrome, obesity (e.g., inflammatory obesity) or diabetes, or a propensity to develop such conditions by detecting an alteration in one or more of adiponectin, insulin, leptin, and CRP in a sample of saliva obtained from the subject relative the level of such biomarkers in a reference. Alterations in the levels of such biomarkers (or any other marker delineated herein) are detected using standard methods. In another approach, diagnostic methods of the invention are used to assay the expression of adiponectin, insulin, leptin, and C-reactive protein (CRP) in a biological sample relative to a reference (e.g., the level of such polypeptides present in a corresponding control sample). In one embodiment, the level of adiponectin, insulin, leptin, and C-reactive protein (CRP) is detected using an antibody that specifically binds the polypeptide. Exemplary antibodies that specifically bind such polypeptides are described herein. Such antibodies are useful for the diagnosis of a metabolic syndrome, obesity (e.g., inflammatory obesity) or diabetes, or a propensity to develop such conditions. Methods for measuring an antibody-marker complex include, for example, detection of fluorescence, luminescence, chemiluminescence, absorbance, reflectance, transmittance, birefringence or refractive index. Optical methods include microscopy (both confocal and non-confocal), imaging methods and non-imaging methods. Methods for performing these assays are readily known in the art. Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Ten, eds., 7th ed. 1991); and Harlow & Lane, supra Immunoassays can be used to determine the quantity of marker in a sample, where an increase in the level of the marker polypeptide is diagnostic of a patient having a metabolic syndrome, obesity (e.g., inflammatory obesity) or diabetes, or a propensity to develop such conditions.

In general, the measurement of a marker polypeptide in a subject sample is compared with a diagnostic amount present in a reference. A diagnostic amount distinguishes between a metabolic syndrome, obesity (e.g., inflammatory obesity) or diabetes, or a propensity to develop such conditions and the absence of such condition. The skilled artisan appreciates that the particular diagnostic amount used can be adjusted to increase sensitivity or specificity of the diagnostic assay depending on the preference of the diagnostician. In general, any significant increase (e.g., at least about 10%, 15%, 30%, 50%, 60%, 75%, 80%, or 90%) in the level of an marker polypeptide or nucleic acid molecule in the subject sample relative to a reference may be used to diagnose a metabolic syndrome, obesity (e.g., inflammatory obesity) or diabetes, or a propensity to develop such conditions. In one embodiment, the reference is the level of marker polypeptide present in a control sample obtained from a patient that does not have a metabolic syndrome, obesity (e.g., inflammatory obesity) or diabetes, or a propensity to develop such conditions. In another embodiment, the reference is a baseline level of marker present in a biologic sample derived from a patient prior to, during, or after treatment for a metabolic syndrome, obesity (e.g., inflammatory obesity) or diabetes, or a propensity to develop such conditions. In yet another embodiment, the reference is a standardized curve.

In another approach, diagnostic methods of the invention are used to assay the expression of adiponectin, insulin, leptin, and C-reactive protein (CRP) in a biological sample relative to a reference (e.g., the level of such polypeptides present in a corresponding control sample). In one embodiment, the level of adiponectin, insulin, leptin, and C-reactive protein (CRP) is detected using an antibody that specifically binds the polypeptide. Exemplary antibodies that specifically bind such polypeptides are described herein. Such antibodies are useful for the diagnosis of a metabolic syndrome, obesity (e.g., inflammatory obesity) or diabetes, or a propensity to develop such conditions. Methods for measuring an antibody-marker complex include, for example, detection of fluorescence, luminescence, chemiluminescence, absorbance, reflectance, transmittance, birefringence or refractive index. Optical methods include microscopy (both confocal and non-confocal), imaging methods and non-imaging methods. Methods for performing these assays are readily known in the art. Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Ten, eds., 7th ed. 1991); and Harlow & Lane, supra Immunoassays can be used to determine the quantity of marker in a sample, where an increase in the level of the marker polypeptide is diagnostic of a patient having a metabolic syndrome, obesity (e.g., inflammatory obesity) or diabetes, or a propensity to develop such conditions.

In general, the measurement of a marker polypeptide in a subject sample is compared with a diagnostic amount present in a reference. A diagnostic amount distinguishes between a metabolic syndrome, obesity (e.g., inflammatory obesity) or diabetes, or a propensity to develop such conditions and the absence of such condition. The skilled artisan appreciates that the particular diagnostic amount used can be adjusted to increase sensitivity or specificity of the diagnostic assay depending on the preference of the diagnostician. In general, any significant increase (e.g., at least about 10%, 15%, 30%, 50%, 60%, 75%, 80%, or 90%) in the level of an marker polypeptide or nucleic acid molecule in the subject sample relative to a reference may be used to diagnose a metabolic syndrome, obesity (e.g., inflammatory obesity) or diabetes, or a propensity to develop such conditions. In one embodiment, the reference is the level of marker polypeptide present in a control sample obtained from a patient that does not have a metabolic syndrome, obesity (e.g., inflammatory obesity) or diabetes, or a propensity to develop such conditions. In another embodiment, the reference is a baseline level of marker present in a biologic sample derived from a patient prior to, during, or after treatment for a metabolic syndrome, obesity (e.g., inflammatory obesity) or diabetes, or a propensity to develop such conditions. In yet another embodiment, the reference is a standardized curve.

Accordingly, a marker profile may be obtained from a subject sample and compared to a reference marker profile obtained from a reference population, so that it is possible to classify the subject as belonging to or not belonging to the reference population. The correlation may take into account the presence or absence of the biomarkers in a test sample and the frequency of detection of the same biomarkers in a control. The correlation may take into account both of such factors to facilitate determination of metabolic syndrome, obesity (e.g., inflammatory obesity) or diabetes, or a propensity to develop such conditions.

Any marker, individually, is useful in aiding in the determination of metabolic syndrome status. First, the selected marker is detected in a subject sample using the methods described herein (e.g. mass spectrometry, immunoassay). Then, the result is compared with a control that distinguishes metabolic syndrome status from non-metabolic syndrome status. As is well understood in the art, the techniques can be adjusted to increase sensitivity or specificity of the diagnostic assay depending on the preference of the diagnostician.

While individual biomarkers are useful diagnostic biomarkers, in some instances, a combination of biomarkers provides greater predictive value than single biomarkers alone. The detection of a plurality of biomarkers (or absence thereof, as the case may be) in a sample can increase the percentage of true positive and true negative diagnoses and decrease the percentage of false positive or false negative diagnoses. Thus, one method provides for the measurement of more than one marker.

### Salivary Glucose Testing

Saliva obtained from pediatric subjects having or at risk of developing a metabolic disease, such as Type 2 diabetes, has altered levels of salivary glucose. In particular, subjects are identified as having a metabolic syndrome, obesity (e.g., inflammatory obesity) or diabetes, or a propensity to develop such conditions by detecting an alteration in glucose in a sample of saliva obtained from the subject relative the level of glucose in a reference. Alterations in the levels of glucose may be detected using methods as described herein or known in the art.

In another approach, diagnostic methods of the invention are used to assay the expression of glucose in a biological sample relative to a reference (e.g., the level of glucose present in a corresponding control sample). In one embodiment, the level of glucose is detected using any of the methods described herein for detection of the other salivary biomarkers.

In one embodiment, the level of glucose is increased relative to a reference. The reference may include, but is not limited to, plasma glucose level, fasting plasma glucose level, salivary glucose level, fasting salivary glucose level, or any standard or control measurement from a healthy control subject, an underweight control subject or a previous level taken from the subject prior to having or being at risk of developing a metabolic disease.

The salivary glucose level may include a ratio of the plasma glucose level. For example, the salivary glucose level may be in a range of about 1:1 to about 1:20 of a plasma glucose level. In an exemplary embodiment, the salivary glucose level is a ratio of about 1:13.5 of a plasma glucose level at plasma glucose concentrations greater than about 80 mg/dL. The ratio may be at least about a 1:1, 1:1.5, 1:2, 1:2.5, 1:3, 1:3.5, 1:4, 1:4.5,1:5, 1:5.5, 1:6, 1:6.5, 1:7, 1:7.5, 1:8, 1:8.5, 1:9, 1:9.5, 1:10, 1:10.5, 1:11, 1:11.5, 1:12, 1:12.5, 1:13, 1:13.5, 1:14, 1:14.5, 1:15, 1:16, 1:17, 1:18, 1:19, 1:20, 1:25, 1:30, 1:35, 1:40, 1:45, 1:50, or greater ratio of a plasma glucose level at plasma glucose concentrations greater than about 70, 71, 72, 73, 74, 75, 75.5, 76, 76.5, 77, 77.5, 78, 78.5, 79, 79.5, 80, 80.5, 81, 81.1, 81.2, 81.3, 81.4, 81.5, 81.6, 81.7, 81.8, 81.9, 82, 82.1, 82.2, 82.3, 82.4, 82.5, 82.6, 82.7, 82.8, 82.9, 83, 83.1, 83.2, 83.3, 83.4, 83.5, 83.6, 83.7, 83.8, 83.9, 84, 84.1, 84.2, 84.3, 84.4, 84.5, 84.6, 84.7, 84.8, 84.9, 85, 85.1, 85.2, 85.3, 85.4, 85.5, 85.6, 85.7, 85.8, 85.9, 86, 86.1, 86.2, 86.3, 86.4, 86.5, 87, 87.5, 88, 88.5, 89, 89.5, 90, 90.5, 91, 91.5, 92, 93, 94, 95 and any number therebetween mg/dL.

In one embodiment, the salivary glucose level is at least about 0.06 mg/dL. The salivary glucose level may be at least about 0.05 mg/dL, 0.06 mg/dL, 0.07, mg/dL, 0.08 mg/dL, 0.09 mg/dL, 0.10 mg/dL, 0.11 mg/dL, 0.12 mg/dL, 0.13 mg/dL, 0.14 mg/dL, 0.15 mg/dL, 0.16 mg/dL, 0.17 mg/dL, 0.18 mg/dL, 0.19 mg/dL, 0.20 mg/dL or greater.

In another embodiment, salivary glucose levels identify pediatric subjects with high plasma glucose levels. The subject may be less than about 18 years of age, such as between about 1 years old and about 18 years old. The subject may be less than about 18, 17, 16, 15, 14, 13, 12, 11, 10, 9, 8, 6, 5, 4, 3, 2, 1 year or less in age.

### Test Device

The test device, such as a lateral flow device, that can take any form desired that provides for the flow of a liquid test sample from the point of contact with the test sample past the test and/or control sites. In general, the test device of the present invention includes an interior flow pathway that includes one or more liquid permeable materials. In a first portion, the device includes a site for the application of a liquid sample. This first portion of the device also includes an analyte-binding conjugate, such as an antibody that specifically binds an antigen of interest (e.g., C-reactive protein (CRP), insulin, glucose, leptin, and adiponectin). The analyte binding conjugate typically binds the analyte to form a complex. Complex formation (e.g., formation of an antigen/antibody conjugate complex) may occur at any point in the interior flow pathway after the analyte contacts the analyte-binding conjugate. For example, complex formation may occur or continue as the sample flows from the first portion to the second portion of the device.

The second portion of the device has a variety of features that enhance functionality. In one embodiment, the second portion is composed of a material capable of filtering the sample to prevent the flow of particulate matter through the device. In another embodiment, the second portion facilitates complex formation by increasing the time required for the liquid to flow from the site of application to the test site. Accordingly, the dimensions of the second portion may be altered (e.g., increased or decreased) to empirically determine for each application those dimensions that enhance sensitivity while reducing false positives, i.e., optimizing the signal-to-noise ratio. In yet another embodiment, the second portion of the device can be used to deliver a desired agent to the liquid as it flows through the device. For example, the second portion may be impregnated with a buffer (e.g., TRIS, sodium carbonate), surfactant (e.g., Tween, Triton), preservative (e.g., Na azide, thimerosol), salt, or other agent, such that contact of the sample with the second portion of the device alters the sample. Exemplary alterations include an increase or decrease in the pH of the sample, in the salt concentration, in the buffering capacity, or in the binding between the conjugate and the analyte (e.g., (e.g., C-reactive protein (CRP), insulin, glucose, leptin, and adiponectin).

The third portion of the device includes a test site, which acts as a readout zone that provides for detection of an analyte in the sample. Various means for detecting the presence of an analyte at a test site are known in the art. In a competitive assay, a labeled probe competes with an analyte of interest for binding to a detector at the test site. The more analyte that is present in the sample, the more effectively it will be able to compete with, and/or displace, the binding of a detector. The hallmark of most competitive assays is that an increase in the amount of analyte in the sample results in a decrease of signal in the readout zone. In contrast, a “sandwich” format typically involves mixing the test sample with a detection probe conjugated with a specific binding member (e.g., antibody). The conjugate and the analyte bind to form a complex. These complexes are then allowed to contact a receptive material (e.g., antibody) that is immobilized at the test site. The analyte/conjugate complex binds to the immobilized receptive material to form a “sandwich complex” (e.g., antibody conjugate/antigen/antibody). In this approach, detection of the “sandwich complex” indicates the presence of analyte in the sample.

It may be desirable to include a positive control to indicate that the liquid sample has traversed the interior flow path from the site of application past the test site. In a competitive assay format, the first portion of the device further includes a control conjugate and the third portion of the device includes a control site with a receptive material that binds the control conjugate. The control site is situated in the third portion of the device downstream from the test site. Detection of control conjugate binding at the control site indicates that the liquid sample flowed from the application site past the test site to the control site. In a sandwich assay format, a control antibody that binds the anti-antigen antibody is fixed at the control site. In the presence or absence of an antigen, excess anti-antigen antibody is detected at the control site.

The device may also include in a fourth portion a wicking pad that contains sorbent material capable of absorbing or adsorbing excess liquid present in the liquid sample.

### Microarrays

The methods of the invention may also be used for microarray-based assays that provide for the high-throughput analysis of biomarkers. The biomarker polypeptides of the invention are useful as hybridizable array elements in such a microarray. The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28:e3.i-e3.vii, 2000), MacBeath et al., (Science 289:1760-1763, 2000), Zhu et al. (Nature Genet. 26:283-289), and in U.S. Pat. No. 6,436,665, hereby incorporated by reference.

Salivary biomarkers: Adiponectin, Resistin, IL-8, VEGF, MCP-1, CRP, Insulin, IL-1β, glucose, MPO, MMP-9, IL-12P70, IL-4, IL-6, IL-13, TNF-alpha, IL-10, IFN-gamma, Leptin, Ghrelin, and IL-17A polypeptides such as those described herein, may also be analyzed using protein microarrays. Typically, protein microarrays feature a protein, or fragment thereof, bound to a solid support. In particular embodiments, the proteins are antibodies that specifically bind a biomarker of the invention (e.g., salivary biomarkers: Adiponectin, Resistin, IL-8, VEGF, MCP-1, CRP, Insulin, IL-1β, glucose, MPO, MMP-9, IL-12P70, IL-4, IL-6, IL-13, TNF-alpha, IL-10, IFN-gamma, Leptin, Ghrelin, and IL-17A). Suitable solid supports include membranes (e.g., membranes composed of nitrocellulose, paper, or other material), polymer-based films (e.g., polystyrene), beads, or glass slides. For some applications, biomarker polypeptides or antibodies recognizing such biomarkers are spotted on a substrate using any convenient method known to the skilled artisan (e.g., by hand or by inkjet printer).

Biomarker levels present in a biological sample taken from a patient, such as a bodily fluid (e.g.,saliva) may be measured using an antibody or other molecule derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

### Selection of Therapies for the Treatment of Metabolic Syndrome or Inflammatory Obesity

Subjects at increased risk for metabolic syndrome, inflammatory obesity, or a propensity to develop a metabolic syndrome, obesity, or diabetes are identified as in need of treatment to ameliorate such conditions or to reduce the risk of developing such conditions. Methods for preventing obesity include, but are not limited to dietary restriction, increased exercise, and the use of appetite suppressants, food intake inhibitors, or other compounds or biological agents useful in reducing obesity.

Examples of food intake inhibitors include but are not limited to gastrointestinal hormone glucagon-like-peptide (Glip-1/Glip-2) and the intestinal preproghrelin derived peptide hormone oxyntomodulin and their analogs, derivatives, mimics Examples of incretins and its agonists, analogs, derivatives, or mimics capable of inducing a decrease in food intake, include but are not limited to GLP-1 receptor agonists exenatide (synthetic mimetic of exendin-4), liraglutide, or enzyme glucagon-like peptide-1 (GLP-1) inhibitors of the dipeptidyl peptidase DPP-4 type, i.e., sitagliptin, vildagliptin, saxagliptin, which slow degradation of GLP-1 and prolong the actions of GLP-1. Such additional anti-metabolic disorder can be administered simultaneously, concurrently or sequentially as the oral compositions or indeed formulated together with the oral compositions.

For subjects identified as having or having a propensity to develop inflammatory obesity or metabolic syndrome are identified as in need of treatment with an anti-inflammatory agent.

### Monitoring

Methods of monitoring metabolic syndrome, obesity (e.g., inflammatory obesity) or diabetes, or a propensity to develop such conditions status in a subject are also useful in managing subject treatment. Provided are methods where the biomarkers (or specific combinations of biomarkers) are measured, such as before and again after subject management or treatment. In these cases, the methods are used to monitor the status of the metabolic syndrome, obesity (e.g., inflammatory obesity) or diabetes, or a propensity to develop such conditions, e.g., response to metabolic syndrome treatment, amelioration of the disease or progression of the disease.

For example, biomarkers (e.g., adiponectin, insulin, leptin, and CRP) can be used to monitor a subject's response to certain treatments of human metabolic syndrome. The level of a marker delineated herein may be measured before treatment, during treatment, or following the conclusion of a treatment regimen. In some embodiments, multiple measurements (e.g., 2, 3, 4, 5) are made at one or more of those times. Measurements are made, for example, using an immunoassay, radioimmunoassay, protein microarray, or other standard method to determine the expression profile of one or more biomarkers (e.g., metabolic and inflammatory proteins). If desired, levels of metabolic or inflammatory biomarkers are compared to reference levels of the metabolic or inflammatory biomarkers to determine if alterations in the metabolic or inflammatory biomarkers are present. Such monitoring may be useful, for example, in assessing the efficacy of a particular treatment in a patient. Therapeutics that normalize the expression of metabolic or inflammatory biomarkers are taken as particularly useful.

### Kits

In one aspect, the invention provides kits for evaluating, such as monitoring the development of or diagnosing, a metabolic syndrome, obesity (e.g., inflammatory obesity) or diabetes, or a propensity to develop such conditions, wherein the kits can be used to detect the biomarkers described herein. For example, the kits can be used to detect any one or more of the biomarkers potentially differentially present in samples of test subjects vs. normal subjects (e.g., adiponectin, insulin, leptin, and CRP) or control proteins. If desired a kit includes any one or more of the following: capture molecules that bind adiponectin, insulin, leptin, CRP, and other metabolic or inflammatory biomarkers. The kits have many applications. For example, the kits can be used to differentiate if a subject has a metabolic syndrome, has a propensity to develop a metabolic syndrome or has a negative diagnosis, thus aiding a metabolic syndrome diagnosis. In another embodiment, kits are provided for aiding the diagnosis of a metabolic syndrome or the diagnosis of a specific type of metabolic syndrome or related condition such as, for example, obesity, diabetes, including Type 1 and Type 2 diabetes, insulin-deficiency, insulin-resistance, insulin-resistance related disorders, glucose intolerance, syndrome X, inflammatory and immune disorders, dyslipidemia, metabolic syndrome, non-alcoholic fatty liver, abnormal lipid metabolism, sleep apnea, hypertension, high cholesterol, atherogenic dyslipidemia, hyperlipidemic conditions such as atherosclerosis, hypercholesterolemia, and other coronary artery diseases in mammals, and other metabolic diseases. The kits can also be used to identify agents that modulate expression of one or more of the herein-described biomarkers in in vitro or in vivo animal models for metabolic syndrome.

The kits may include instructions for the assay, reagents, testing equipment (test tubes, reaction vessels, needles, syringes, etc.), standards for calibrating the assay, and/or equipment provided or used to conduct the assay. The instructions provided in a kit according to the invention may be directed to suitable operational parameters in the form of a label or a separate insert.

Optionally, the kit may further comprise a standard or control information so that the test sample can be compared with the control information standard to determine if the test amount of a marker detected in a sample is a diagnostic amount consistent with a diagnosis of metabolic syndrome, obesity (e.g., inflammatory obesity) or diabetes, or a propensity to develop such conditions.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1

### Saliva of Obese Children Showed Imbalances in Critical Immunometabolic Factors

When used to study development of disease from childhood, the advantage of non-invasive sample collection becomes a major consideration. Since many blood elements partition into saliva, the study of blood elements that occur in saliva has attracted considerable interest. Saliva samples were collected from 8,319 10-12-year old Kuwaiti children. It is well known that this population has a high risk for becoming obese and developing Type 2 diabetes. The relationship between insulin-resistant obesity and systemic elevation of pro-inflammatory cytokines supports the result that saliva of obese children showed imbalances in critical immunometabolic factors. In a random subset of 744 children, salivary levels of 20 hormones and cytokines were measured. These biomarkers include Adiponectin, Resistin, IL-8, VEGF, MCP-1, CRP, Insulin, IL-1β, MPO, MMP-9, IL-12P70, IL-4, IL-6, IL-13, TNF-alpha, IL-10, IFN-gamma, Leptin, Ghrelin, and IL-17A. The results of this study provide an early insight into the development of a metabolic disease in children and establish that non-invasive methods are robust and useful for data collection in studies of vulnerable subjects.

### Example 2

### Salivary Insulin, CRP, Leptin, and Adiponectin Levels were Altered in Obese Children Relative to Children of Normal Weight

Saliva samples (744) were randomly selected from 8,319 saliva samples collected from children to provide 186 for each category of underweight, normal healthy weight, overweight and obese. Except for the underweight category, where inadequate male samples were available, each body weight category was filled with equal numbers of boys and girls. Characteristics of these groups are evaluated in Table 1 (below).

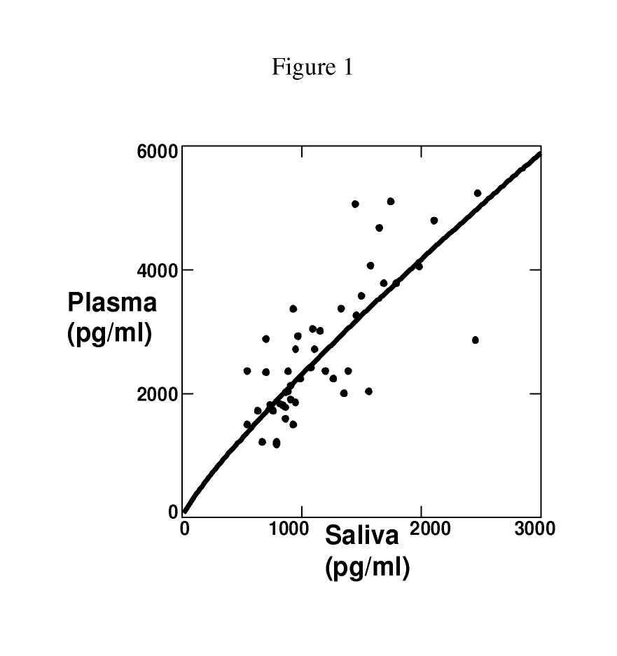

No significant differences in age between groups were noted. Body mass index (BMI), waist circumference and systolic blood pressure exhibited significant incremental increase as the body weight category moved to obese. In every body weight category, BMI and waist circumference significantly differed from each other. Systolic and diastolic blood pressure were not significantly different between underweight and normal children. Systolic and diastolic blood pressure in otherwise normal, overweight and obese children were all significantly different from each other. Obese children had significantly lower fitness as measured by exercise stimulated heart rate elevation than any other category. Obese 11-year old Kuwaiti children had 64% higher BMI, 40% greater waist circumference and 17% higher systolic and diastolic blood pressure and 50% greater exercise elevation of heart rate as a measure of unfitness compared to normal weight children. Table 2 shows similar statistics from 53 US children used to test the relative concentration of insulin in saliva and blood (FIG. 1).

TABLE 1
Age, BMI, waist circumference and systolic blood pressure of 744 Kuwaiti children (mean ± S.D.). Significance tested as
pooled male and female subjects in each body weight category for p < 0.001, the Bonferonni correction for 36 comparisons.

| | Probe | Age | | BMI | | Waist | | Systolic BP | | Diastolic BP | | Fitness | |
|---|---|---|---|---|---|---|---|---|---|---|---|---|---|
| | Units | years | p | kg/m² | p | cm | p | mmHg | p | mmHg | p | beats/min. | p |
| 1)Under- | M(64) | 11.76 ± 0.43 | NS | 14.13 ± 0.64 | *2, 3, | 60.48 ± 54.1 | *2, 3, | 97.18 ± 10.75 | *3, 4 | 67.25 ± 10.95 | *3, 4 | 18.81 ± 17.24 | *4 |
| weight | F(122) | 11.51 ± 0.58 | | 13.86 ± 0.71 | 4 | 54.9 ± 6.03 | 4 | 100.92 ± 14.85 | | 68.66 ± 12.43 | | 22.77 ± 19.43 | |
| (186) | | | | | | | | | | | | | |
| 2) Normal | M(93) | 11.52 ± 0.62 | NS | 17.46 ± 1.50 | *1, 3, | 58.5 ± 4.66 | *1, 3, | 100.53 ± 12.92 | *3, 4 | 68.72 ± 12.01 | *3, 4 | 18.06 ± 18.09 | *4 |
| (186) | F(93) | 11.39 ± 0.59 | | 17.73 ± 1.88 | 4 | 61.64 ± 6.29 | 4 | 104.54 ± 15.26 | | 70.74 ± 11.15 | | 26.58 ± 23.99 | |
| 3) Over- | M(93) | 11.55 ± 0.56 | NS | 22.12 ± 0.97 | *1, 2, | 70.19 ± 5.11 | *1, 2, | 112.74 ± 13.51 | *1, 2, | 74.94 ± 11.49 | *1, 2, | 23.7 ± 18.48 | *4 |
| weight | F(93) | 11.44 ± 0.58 | | 22.86 ± 1.10 | 4 | 72.59 ± 5.67 | 4 | 114.47 ± 13.65 | 4 | 77.23 ± 12.98 | 4 | 24.68 ± 19.86 | |
| (186) | | | | | | | | | | | | | |
| 4) Obese | M(93) | 11.50 ± 0.50 | NS | 28.79 ± 4.14 | *1, 2, | 83.49 ± 9.47 | *1, 2, | 122.37 ± 18.24 | *1, 2, | 82.18 ± 14.09 | *1, 2, | 31.89 ± 21.82 | *1, |
| (186) | F(93) | 11.43 ± 0.57 | | 28.8 ± 3.62 | 3 | 84.69 ± 8.06 | 3 | 118.22 ± 14.49 | 3 | 81.39 ± 13.63 | 3 | 35.17 ± 19.47 | 2, 3 |

NS = not significant when compared with any other group.
* = significant (p < 0.001) differences between the category and the numbers representing other categories.

TABLE 2
Age, BMI, waist circumference and systolic blood pressure of 53 U.S. children (mean ± S.D) used to
determine the saliva and plasma calibration curve (FIG. 1).

| | Probe | Age | BMI | Waist | Systolic BP | Diastolic BP | Unfitness |
|---|---|---|---|---|---|---|---|
| | Units | years | kg/m² | cm | mmHg | mmHg | beats/minute |
| Normal | M(14) | 10.1 ± 1.14 | 17.1 ± 1.77 | 61.32 ± 5.98 | 117.59 ± 9.57 | 68.33 ± 8.46 | 22.58 ± 15.21 |
| (25) | F(11) | 10.21 ± 1.09 | 17.15 ± 2.32 | 61.88 ± 6.35 | 118.39 ± 10.14 | 68.09 ± 5.74 | 17.6 ± 17.52 |
| Overweight | M(5) | 10.97 ± 1.07 | 21.71 ± 1.07 | 76.67 ± 8.05 | 125.06 ± 8.44 | 71.26 ± 4.03 | 28.3 ± 13.77 |
| (8) | F(3) | 11.3 ± 1.73 | 23.66 ± 1.99 | 77.89 ± 13.98 | 125.22 ± 13.96 | 69.44 ± 6.55 | 24.00 ± 22.50 |
| Obese | M(14) | 11.94 ± 2.18 | 31.93 ± 7.97 | 100.77 ± 18.35 | 127.04 ± 9.87 | 71.14 ± 8.58 | 32.96 ± 8.97 |
| (20) | F(6) | 10.25 ± 1.56 | 26.77 ± 3.73 | 80.75 ± 8.89 | 125.44 ± 11.39 | 68.38 ± 8.61 | 54.8 ± 36.06 |

Twenty salivary biomarkers: Adiponectin, Resistin, IL-8, VEGF, MCP-1, CRP, Insulin, IL-1β, MPO, MMP-9, IL-12P70, IL-4, IL-6, IL-13, TNF-alpha, IL-10, IFN-gamma, Leptin, Ghrelin, and IL-17A were selected to evaluate metabolic changes associated with obesity in 11-year old children (n=744) in the four body weight categories; underweight, normal healthy weight, overweight and obese. The assay performance is summarized in Table 3. The median concentration of 17 biomarkers was greater than the manufacturer's stated assay sensitivity. For three other biomarkers (IL-10, leptin and ghrelin) median values fell below the assay sensitivity. The analysis software allowed extrapolation beyond the lowest standard provided when the value did not fall on or below the blank. Using this feature provided a means to order low concentrations for non-parametric rank analysis. All probes provided non-zero values for 95 to 100% of the samples tested except for leptin. Approximately 37.4% of the leptin determinations were too low to measure. In the leptin analysis, however, the percentage of measurable samples increased with increasing obesity so that analysis by non-parametric rank could be performed without introducing bias.

TABLE 3
Salivary concentration, manufacturer's stated assay sensitivity, assay precision and
lowest assay standard of 20 biomarkers measured by multiplex assay. Three biomarkers,
IL-10, leptin and ghrelin had median concentrations less than the assay sensitivity.

| | | | | | Precision | Precision | | |
|---|---|---|---|---|---|---|---|---|
| | Median | | | Assay | Intra- | Inter- | lowest | Median |
| | Conc. | 25^{th} | 75^{th} | Sensitivity | Assay | Assay | std | Conc. < |
| Biomarker | (pg/ml) | Percentile | Percentile | (pg/ml) | (CV %) | (CV %) | (pg/ml) | Sen. |
| Adiponectin | 3445.83 | 1900.66 | 6491.58 | 19.80 | 4.50 | 12.3 | 16.78 | 0 |
| Resistin | 1669.57 | 937.35 | 2783.92 | 7.30 | 7.90 | 9.3 | 1.71 | 0 |
| IL-8 | 468.85 | 250.02 | 1017.41 | 0.40 | 1.90 | 3.5 | 0.12 | 0 |
| VEGF | 290.34 | 205.37 | 417.74 | 26.30 | 3.70 | 10.4 | 18.87 | 0 |
| MCP-1 | 255.77 | 170.93 | 397.44 | 1.90 | 1.50 | 7.9 | 0.87 | 0 |
| CRP | 165.88 | 49.65 | 451.44 | 1.90 | 6.40 | 10.0 | 1.33 | 0 |
| Insulin | 59.15 | 27.52 | 120.70 | 58.00 | 3.00 | 6.0 | 3.30 | 0 |
| IL-1β | 45.43 | 19.22 | 111.53 | 0.80 | 2.30 | 6.7 | 0.12 | 0 |
| MPO | 30.68 | 22.26 | 40.48 | 7.00 | 12.30 | 16.3 | 0.12 | 0 |
| MMP-9 | 19.50 | 9.70 | 43.20 | 1.00 | 6.80 | 11.7 | 0.02 | 0 |
| IL-12P70 | 13.20 | 4.66 | 34.47 | 0.60 | 2.20 | 16.7 | 0.63 | 0 |
| IL-4 | 7.38 | 3.40 | 15.80 | 4.50 | 2.90 | 14.2 | 0.68 | 0 |
| IL-6 | 7.08 | 3.49 | 14.84 | 0.90 | 2.00 | 18.2 | 0.63 | 0 |
| IL-13 | 6.61 | 3.10 | 12.66 | 1.30 | 2.20 | 9.2 | 0.08 | 0 |
| TNF-α | 6.14 | 3.05 | 11.95 | 0.70 | 2.60 | 13.0 | 0.13 | 0 |
| IL-10 | 4.69 | 2.59 | 8.09 | 8.60 | 1.60 | 16.8 | 0.11 | 1 |
| IFN-γ | 2.16 | 0.87 | 4.95 | 0.80 | 1.60 | 12.0 | 0.06 | 0 |
| Leptin | 1.29 | 0.01 | 4.92 | 27.00 | 3.00 | 4.0 | 5.02 | 1 |
| Ghrelin | 1.14 | 1.14 | 6.28 | 2.00 | 2.00 | 8.0 | 5.29 | 1 |
| IL-17A | 0.97 | 0.00 | 3.17 | 0.70 | 2.20 | 7.9 | 0.12 | 0 |

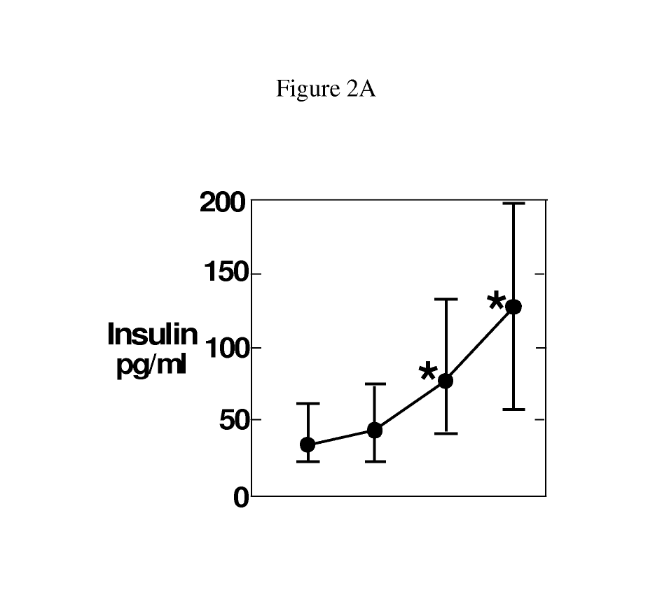

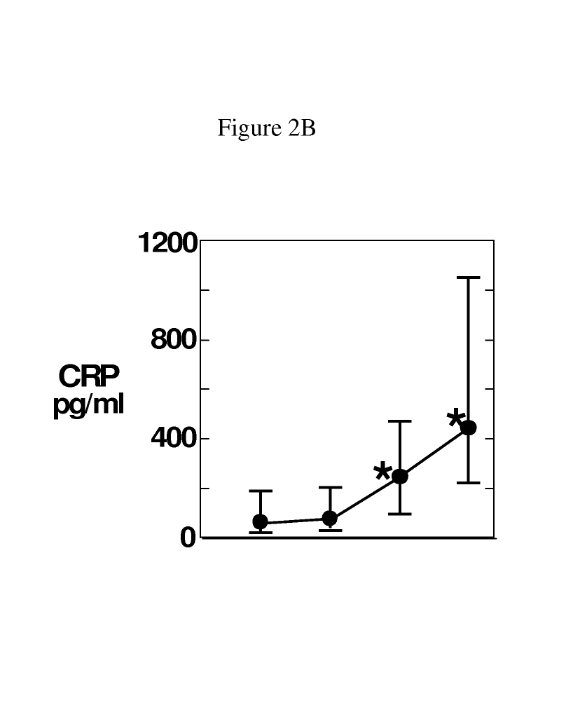

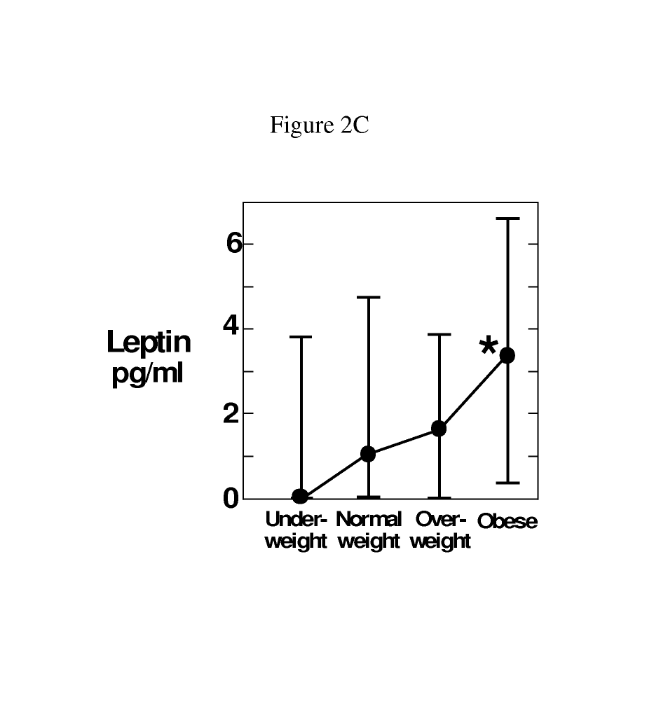

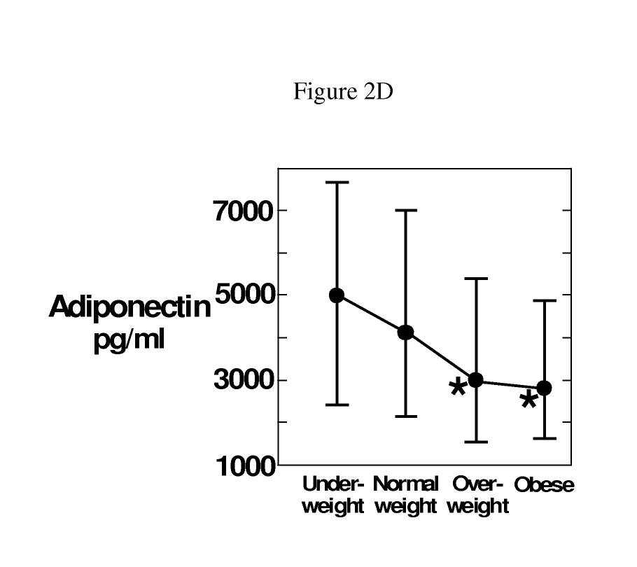

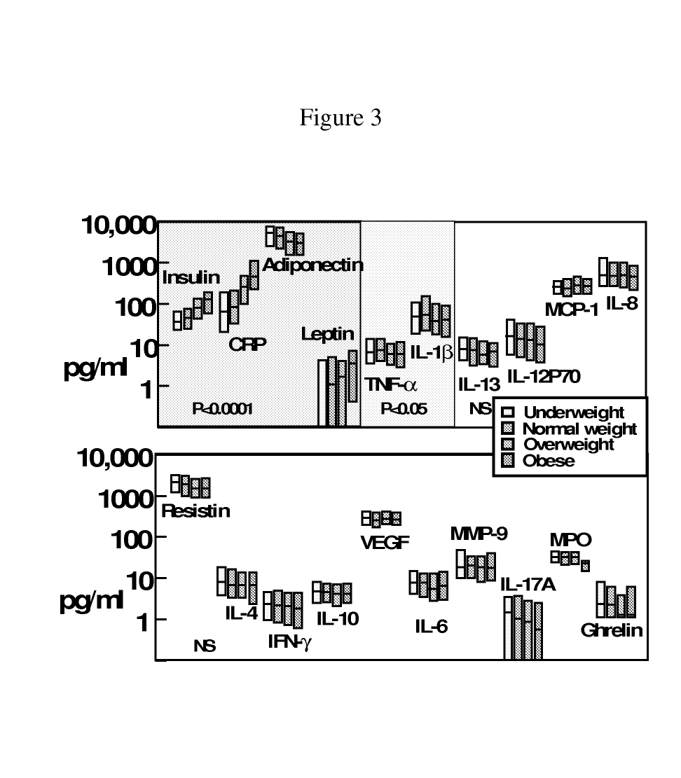

The assay results are summarized in FIGS. 2A-2D and Table 4. Of the 20 probes tested, significant differences between obese and normal weight children occurred in the concentration of insulin (FIG. 2A), CRP (FIG. 2B), leptin (FIG. 2C) and adiponectin (FIG. 2D). As indicated in Table 4, salivary insulin levels in obese children (median=127 pg/ml) were almost 3 times that of normal weight children (median=44 pg/ml) and salivary CRP of obese children (median=435 pg/ml) was almost 6 times that of normal weight children (median=76 pg/ml). The estimated concentration of salivary leptin in obese children (median=3.3 pg/ml) was 3 times that of normal weight children (1 pg/ml). Salivary adiponectin decreased by approximately 30% with increasing obesity from that of normal healthy weight children(median 4,083 pg/ml) to overweight and obese children (median=2981 and 2798 pg/ml respectively). There were no significant differences between biomarker concentrations of underweight and normal healthy weight children. Salivary levels of the remaining 16 probes tested did not significantly change when analyzed by body weight categories. These are illustrated in FIG. 3 and tabulated in Table 5.

TABLE 4
Concentration of insulin, c-reactive protein (CRP), adiponectin and leptin in saliva of
children by body weight category and gender. Summary statistics are median, interquartile
range (N subjects). Values recorded for sixteen additional probes tested are in supplement
Table 2. Probability levels for age, gender, overweight, obese and underweight were computed
by Wilcoxon regression relative to normal healthy weight children.

| | Probe | Insulin | CRP | Adiponectin | Leptin |
|---|---|---|---|---|---|
| | Units | pg/ml | pg/ml | pg/ml | pg/ml |
| Underweight (186) | M(64) | 39.17, 48.63 | 56.64, 92.32 | 4420.69, 6424.31 | 0.01, 5.31 |
| | F(122) | 34.30, 38.02 | 61.99, 182.74 | 5059.84, 4573.13 | 0.01, 3.22 |
| Normal (186) | M(93) | 39.39, 45.38 | 73.01, 153.75 | 4220.48, 5303.44 | 1.06, 4.77 |
| | F(93) | 44.70, 54.38 | 77.15, 186.95 | 3993.59, 5051.76 | 0.63, 4.61 |
| Overweight (186) | M(93) | 80.39, 88.74 | 177.46, 311.93 | 2401.50, 3785.25 | 1.06, 3.26 |
| | F(93) | 76.25, 87.13 | 281.39, 516.54 | 3321.79, 3693.02 | 2.41, 5.03 |
| Obese (186) | M(93) | 112.98, 125.09 | 429.44, 668.52 | 2547.76, 2778.61 | 3.16, 6.4 |
| | F(93) | 143.50, 150.24 | 443.13, 1033.29 | 3061.71, 3752.00 | 3.70, 6.41 |
| Wilcoxon regression p | Age | 0.107 | 0.083 | 0.373 | 1.000 |
| | Gender | 0.337 | 0.028 | 0.471 | 1.000 |
| | Overweight | <0.0001 | <0.0001 | 0.001 | 0.408 |
| | Obese | <0.0001 | <0.0001 | <0.0001 | <0.0001 |
| | Underweight | 0.157 | 0.266 | 0.142 | 1.000 |

TABLE 5
Concentration of sixteen cytokines in saliva of Kuwaiti children by body weight category and gender. Summary statistics are
median, interquartile range (N subjects). Probability levels for overweight, obese and underweight were computed by Wilcoxon
regression relative to normal healthy weight children.

| | | Units | | | | | | | |
|---|---|---|---|---|---|---|---|---|---|
| | | pg/ml | pg/ml | pg/ml | pg/ml | pg/ml | pg/ml | pg/ml | pg/ml |
| | | Probe | | | | | | | |
| | | TNF-α | IL-1β | IL-13 | IL-12P70 | MCP-1 | IL-8 | Resistin | IL-4 |
| Underweight | M(64) | 5.56, 13.12 | 41.79, 155.69 | 5.78, 9.22 | 12.74, 40.70 | 219.10, 223.40 | 492.90, 1333.8 | 1949.97, 2476.35 | 7.45, 17.11 |
| (186) | F(122) | 6.60, 9.25 | 51.64, 74.31 | 8.09, 10.49 | 18.92, 34 | 255.00, 152.60 | 492.00, 713.40 | 2105.99, 1965.13 | 8.70, 15.60 |
| Normal | M(93) | 7.29, 10.92 | 63.54, 161.92 | 6.90, 10.19 | 18.23, 38.11 | 279.20, 321.80 | 530.70, 868.20 | 1913.35, 2254.60 | 7.19, 18.41 |
| (186) | F(93) | 6.52, 9.36 | 41.90, 94.12 | 7.39, 10.74 | 11.71, 22.78 | 207.00, 185.00 | 445.40, 615.00 | 1620.63, 1722.53 | 7.16, 12.06 |
| Overweight | M(93) | 6.10, 7.75 | 45.11, 94.71 | 5.69, 7.19 | 12.65, 29.24 | 301.20, 320.30 | 545.30, 876.50 | 1309.83, 1317.71 | 7.19, 10.33 |
| (186) | F(93) | 5.08, 6.94 | 32.92, 83.4 | 5.32, 8.92 | 13.92, 27.40 | 259.80, 235.40 | 394.20, 656.80 | 1552.14, 2011.18 | 6.79, 11.39 |
| Obese | M(93) | 5.31, 9.25 | 44.21, 91.45 | 6.62, 7.08 | 9.80, 25.19 | 262.90, 234.00 | 437.20, 612.50 | 1245.30, 1789.36 | 8.43, 13.54 |
| (186) | F(93) | 6.17, 8.25 | 35.86, 64.23 | 5.89, 8.97 | 10.78, 25.07 | 261.60, 188.90 | 452.20, 611.10 | 1816.73, 1661.42 | 5.85, 11.19 |
| Wilcoxon | Age | 0.979 | 0.607 | 0.462 | 0.589 | 0.724 | 0.676 | 0.661 | 0.974 |
| regression p | Gender | 0.195 | 0.130 | 0.390 | 0.343 | 0.033 | 0.313 | 0.030 | 0.878 |
| | Overweight | 0.040 | 0.061 | 0.193 | 0.540 | 0.035 | 0.691 | 0.103 | 0.320 |
| | Obese | 0.019 | 0.035 | 0.093 | 0.102 | 0.142 | 0.167 | 0.185 | 0.215 |
| | Underweight | 0.467 | 0.492 | 0.524 | 0.225 | 0.638 | 0.593 | 0.312 | 0.385 |
| | | Probe | | | | | | | |
| | | IFN-γ | IL-10 | VEGF | IL-6 | MMP-9 | IL-17A | MPO | Ghrelin |
| Underweight | M(64) | 2.50, 4.71 | 5.05, 6.34 | 318.70, 249.50 | 7.64, 12.50 | 15.30, 31.61 | 1.31, 5.15 | 28.11, 21.23 | 2.72, 7.68 |
| (186) | F(122) | 2.41, 3.85 | 5.14, 5.69 | 306.00, 4573.13 | 9.12, 10.75 | 23.04, 41.24 | 1.47, 3.20 | 32.40, 25.10 | 1.36, 5.95 |
| Normal | M(93) | 2.21, 5.81 | 5.84, 5.44 | 321.00, 281.10 | 9.38, 14.89 | 22.52, 33.57 | 0.93, 4.14 | 28.76, 16.11 | 2.72, 5.36 |
| (186) | F(93) | 2.21, 2.8 | 3.85, 4.56 | 233.40, 163.50 | 7.23, 8.87 | 19.37, 30.22 | 0.97, 2.79 | 32.66, 21.30 | 1.14, 3.72 |
| Overweight | M(93) | 2.32, 3.48 | 4.23, 5.51 | 320.60, 290.50 | 6.38, 10.54 | 17.82, 25.16 | 0.84, 2.86 | 31.75, 14.19 | 1.14, 3.34 |
| (186) | F(93) | 2.02, 4.16 | 4.82, 5.02 | 265.00, 139.70 | 5.40, 11.74 | 19.80, 32.95 | 0.95, 3.02 | 30.96, 19.65 | 1.14, 1.78 |
| Obese | M(93) | 1.93, 4.48 | 4.57, 5.02 | 329.00, 294.40 | 5.85, 13.10 | 15.32, 25.71 | 0.70, 2.80 | 28.78, 14.46 | 1.24, 5.14 |
| (186) | F(93) | 1.67, 3.92 | 4.44, 5.80 | 242.80, 175.00 | 7.69, 11.20 | 21.14, 47.36 | 0.49, 2.61 | 30.52, 16.34 | 1.14, 5.49 |
| Wilcoxon | Age | 0.837 | 0.244 | 0.310 | 0.357 | 0.229 | 1.000 | 0.010 | 1.000 |
| regression p | Gender | 0.147 | 0.052 | 0.000 | 0.737 | 0.080 | 1.000 | 0.090 | 1.000 |
| | Normal | 0.530 | 0.143 | 0.310 | 0.126 | 0.477 | 0.747 | 0.582 | 1.000 |
| | Overweight | 0.238 | 0.391 | 0.560 | 0.609 | 0.612 | 0.747 | 0.801 | 1.000 |
| | Obese | 0.546 | 0.680 | 0.094 | 0.367 | 0.736 | 0.089 | 0.252 | 1.000 |

Salivary C-reactive protein (CRP) was almost 6 times higher, and salivary insulin and leptin were approximately 3 times higher (all p<0.0001) in obese compared to healthy normal weight children. Adiponectin was approximately 30% lower in obese children (p<0.0001).

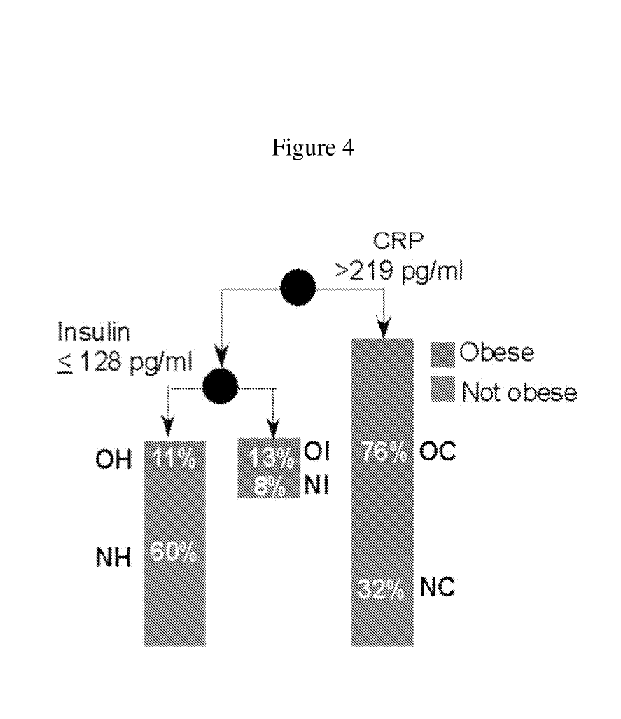

The diagnostic implications of these findings were evaluated by classification tree topology in FIG. 4. Approximately 76.3% of the obese children were identified as having >219 pg/ml CRP in their saliva, indicating that the inflammatory state was the most common form of obesity in children. Of the obese children with lower levels of CRP, 13% had high insulin (>128 pg/ml) and 11% had low insulin. Using only salivary CRP and insulin as predictors, the overall diagnostic sensitivity for identifying obesity was 89% and specificity was 61%.

The correspondence between saliva and plasma concentration of insulin were evaluated in a smaller (N=53) population (FIG. 1) from the United States. This comparison was between saliva and plasma samples both determined by a multiplexed quantitation and detection assay. The result indicated that the immunoreactive saliva insulin concentration was approximately half of the plasma concentration and a reasonable estimate of plasma insulin with high correlation. By this approximation, the predictor variable of 128 pg/ml salivary insulin was equivalent to 68 pmoles/L (11 μU/ml) in plasma, a value higher than that reported for normal fasting children (38-46 pmoles/L=6.3-7.6 μU/ml).

Investigation of the properties of each of the six groups described by the categorization tree revealed significant differences relative to the normal healthy (NH) group in both clinical measures and biomarker concentrations (Table 6). Systolic blood pressure, BMI, waist circumference and insulin were significantly elevated in all categories except for the non-obese healthy group (NH). Diastolic blood pressure was significantly elevated in the obese with high insulin (OI) and obese with high CRP (OC) groups. Low fitness was increased in the OH group and significantly increased in the OC group. CRP and IL-6 were significantly elevated in both high CRP groups (OC and NC) and leptin was significantly elevated in the obese high CRP group (OC). The non-obese, high CRP group (NC) exhibited elevation of multiple biomarkers (IL-10, resistin, IL-1β and MMP-9). The obese but healthy group (OH) exhibited significantly lowered levels of IL-10 and adiponectin.

TABLE 6
Median values for measures and biomarkers of groups defined by CRP and insulin
concentration.

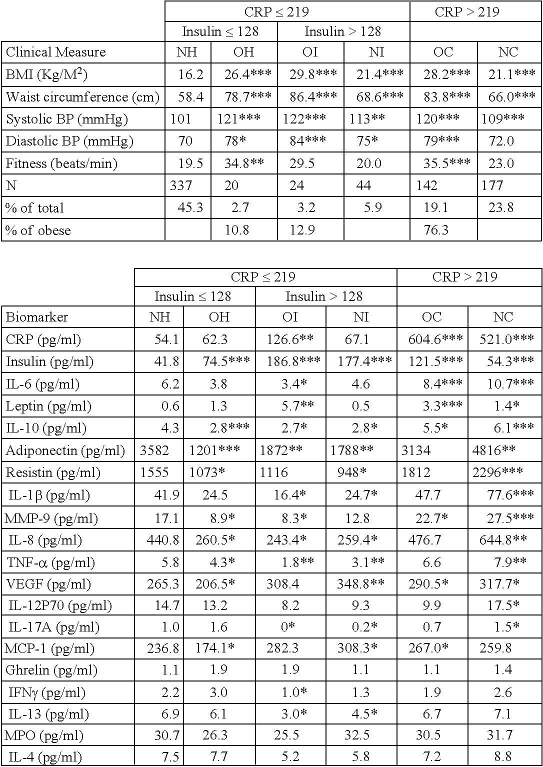

***p < 0.0004
**p < 0.001
*p < 0.05
Relative to NH:

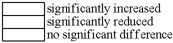

Many elements found in blood also occur in saliva with their concentration levels often correlating. This association has been used effectively in the study of cortisol metabolism for many years. In the study described herein, 20 possible biomarkers related to obesity were surveyed and four were found that exhibit significant changes with increasing body weight in a pediatric population. These data suggest that saliva could be a useful blood surrogate for the study of obesity in populations such as children, the elderly or traditionally underserved minority adults, for whom repeated blood sampling can be both traumatic and difficult.

Elevated plasma insulin is a primary characteristic of Type 2 diabetes and also is proportional to body fat content. Salivary insulin exhibits a positive linear relation with plasma insulin during the glucose tolerance test and correlates well with plasma concentration after insulin injection. Plasma insulin decreases in parallel with weight loss in obese children enrolled in a study of therapy by lifestyle change.

The greatest change measured was that of salivary CRP concentrations which has also been found to significantly correlate with serum concentrations. In vitro and small animal studies suggest that high blood CRP may cause insulin resistance by increasing insulin receptor substrate (IRS)-1 phosphorylation. Human studies associate high levels of CRP with metabolic syndrome and Type 2 diabetes. Isolated adipocyte studies suggest that high CRP suppresses adiponectin synthesis which could explain the reduced salivary adiponectin levels seen in study. Analysis suggests that hypoadiponectinemia can be an independent risk factor for metabolic syndrome. Salivary adiponectin has been reported to significantly correlate with plasma adiponectin levels. Blood levels of leptin can also be proportional to body fat content highly correlated to salivary leptin concentrations, which are about one-fourth the concentration in plasma.

There does not appear to be any data to suggest that salivary levels of the biomarkers measured have any effect on the oral cavity or gastrointestinal tract. However, there is evidence that leptin may have an effect on the modulation of taste receptors on the tongue. Salivary leptin is produced, stored and secreted by salivary glands and has been detected in salivary gland ductal tissue by immunochemistry. Taste receptors mediating the sensation of sweetness are localized to tip of the tongue and along the lateral border of the anterior ⅔ of the tongue. The sensation is conducted by the chorda tympani division of the facial (7^{th} cranial) nerve to the nucleus tractus solitaries (NTS) in the hypothalamus.

Neurophysiological facial nerve recordings of applied sucrose following leptin administration to the tongue in mice demonstrated that leptin reduced sweet receptor sensitivity. In a study of taste preferences in experimental animals, obese rats had high leptin blood levels and reduced preference for sucrose compared to normal weight animals. Taken together with the data herein, which demonstrated elevated salivary leptin in obese children (FIG. 2C), it may be reasonable to suggest that sweet receptor sensitivity is reduced in obese children by their high salivary leptin concentration which could also account for the reduced dental decay observed in obese children.

The first level of the classification tree (CRP>219 pg/ml, FIG. 1), clearly associated with inflammatory mediators, identified two groups of children. The obese high salivary CRP (OC), represented the largest group of obese children (76%). The non-obese high salivary CRP group (NC) was numerically larger. Comparing their individual group characteristics (Table 6), differences between these groups were elevated diastolic blood pressure, significantly reduced fitness and elevated leptin in the obese (OC) group. The non-obese high salivary CRP group (NC) had high levels of CRP, insulin, IL-6 and four other inflammatory mediators (IL-10, resistin, IL-1b, and MMP-9), with elevated adiponectin as an anti-inflammatory mediator.

The second level of the classification tree (CRP≦219, Insulin >128 pg/ml, FIG. 1) also identified two groups of children (OI and NI), but these did not have elevated salivary inflammatory mediators. However, these children but did have extremely elevated insulin levels. The principle differences were that obese children had high systolic and diastolic blood pressure. Mediator levels of both CRP and leptin were elevated in the obese group (OI) but not the non-obese group (NI). Adiponectin was reduced in both.

Analysis suggests that there may be three types of obesity in children. Approximately 76% of obese children had high (>219 pg/ml) CRP, with a decidedly inflammatory character. In the children with CRP≦128 pg/ml, 13% of the obese children had high salivary insulin but no elevated inflammatory mediators and the remaining 11% obese children had only slightly elevated salivary insulin but significantly reduced adiponectin. In addition, 40% of the non-obese children were found in groups which, based on biomarker characteristics, seemed to be at risk for becoming obese.

Children with both low insulin and low CRP included a normal healthy group (NH) and an obese group (OH). In this case, obese children were associated with slightly but significantly elevated insulin, reduced adiponectin, IL-10 and fitness.

Comparison of the observations herein with reported immunohistochemistry of obese adult adipose tissue (J Am Coll Cardiol 2011; 58:232-7) strongly suggested that a group within obese adults (represented by CLS+ macrophage crown-like structure positive and approximately 72% of obese adults) may coincide with the children designated as CRP>219 in the data herein (76.3% of obese children). The studies herein suggest, however, that cohorts with comparable biomarker levels exist that are not obese which could be at risk for obesity development.

The constellation of biomarkers that appear in saliva are clearly a reflection of underlying pathology. High levels of salivary CRP, myoglobin and MPO, for example appear following myocardial infarction. Periodontal disease characteristically exhibits high levels of salivary MMP-9 and IL-1β. Inflammatory bowel disease is associated with elevated salivary IL-6 and CRP. Without being bound to any particular theory, the data herein suggests that adolescent obesity is associated with high levels of salivary insulin and CRP.

Novel, robust and non-invasive measures of immunometabolic parameters are needed to support efforts to stratify risks for obesity-associated co-morbidities and for medical management, particularly among at-risk populations where traditional biochemical assays of blood and tissue samples are difficult to obtain. The identification of four salivary biomarkers in 11-year old children that significantly change with increasing obesity make use of relatively non-invasive biomarkers, particularly in longitudinal studies, to investigate development of metabolic diseases in children and evaluate therapeutic interventions. In addition, the observation of elevated salivary leptin levels in obese children provides analytical support for the hypothesis that salivary leptin serves an exocrine function in which the mechanism of action is regulation of tongue sweet receptor response.

The results of this study suggest that obesity may be characterized and classified by salivary biomarker levels. While not being bound to any particular theory, these biomarkers may offer potential for non-invasive investigation of changes that lead to advanced stages of metabolic disease, such as Type 2 diabetes, and evaluation of therapeutic procedures for prevention of obesity in vulnerable populations.

### Example 3

### Salivary Glucose Concentration Exhibits Threshold Kinetics in Normal-Weight, Overweight, and Obese Children

The present investigation into the correlation between salivary and plasma glucose levels in children has been prompted by the need for a non-invasive method to determine the occurrence of hyperglycemia as part of the syndrome describing metabolic disease. Fasting plasma and salivary glucose concentrations in a sample of 65 US children with a mean age of 10.6±0.2 y were compared and it was found that salivary glucose levels exhibit threshold kinetics. While plasma glucose concentrations less than the threshold value of 84.8 mg/dL produced unmeasurable salivary glucose levels, plasma concentrations over the threshold appeared to produce a linear rise in salivary glucose levels. It is well known that hyperglycemia (fasting blood glucose ≧100 mg/dL) occurs in children at a frequency of up to 10%, and the development of hyperglycemia is clearly an important step in the progression of metabolic syndrome to Type 2 diabetes. Prior data suggest that salivary glucose testing may be a potentially useful screening tool for metabolic syndrome in children. While salivary glucose testing may miss up to 50% of children with high plasma glucose levels, it would almost certainly identify those children who do not have high plasma glucose levels, sparing these children from further invasive testing. This method also has the advantage of being free of adverse reactions, compared to an adverse reaction incidence of 2% and a loss-of-consciousness incidence of 0.3% reported in a study involving venous sampling in children.

The identification of a threshold response in human salivary glucose concentration is intriguing. The phenomenon of a salivary glucose concentration threshold was first reported by investigators studying canine saliva. As early as 1891, these investigators reported that glucose does not normally appear in the saliva of dogs, but when the blood glucose concentration was elevated by intravenous infusion to approximately 512 mg/dL, glucose began to appear in saliva at a level proportional to blood levels. Data indicate that salivary glucose in humans also exhibits a threshold response (Table 7). The data suggest that the human salivary glucose threshold is in the range of 84.8 to 136.8 mg/dL and that the slope is in the range of 6.4 to 55.3. It is clear that these values may vary depending of the glandular source of saliva, the method of collection, the population characteristics, and even the individual subject. This latter point was made elegantly in a study that continuously measured salivary and blood glucose levels in 6 normal-weight adult subjects following the oral administration of 75 g of glucose. Considerable variability existed between individuals in both the salivary threshold and the slope of the blood: saliva concentration response function. Therefore, if salivary glucose testing were to be adopted clinically, a standardized protocol for fasting, collection, and analysis would need to be established.

A threshold plasma level for the appearance of glucose in saliva evokes the relationship between plasma and renal glucose concentrations. The data in Table 7 include an estimated threshold response in urine of 152.7 mg/dL27, suggesting that salivary glands exhibit a lower glucose threshold than does the kidney. There are several similarities between the kidney and salivary glands. Morphologic and immunologic similarities between the striated ducts of the salivary gland and the kidney have been demonstrated. For example, in animal models, the Na(+)-dependent glucose co-transporter SGLT1 was found in both the kidney and in the acinar and ductal cells of salivary glands. As with urine in the kidney, the formation of saliva has been proposed to be a 2-stage process. In this model, saliva was initially formed by salivary acinar cells as a primary fluid with a small-molecule composition similar to that of plasma. The primary fluid was then modified by the salivary gland system that reabsorbed sodium and glucose until the resulting hypotonic glucose-depleted (relative to plasma) oral saliva was secreted into the mouth. It seems possible that the salivary glucose threshold could occur by a mechanism similar to that found in the kidney.

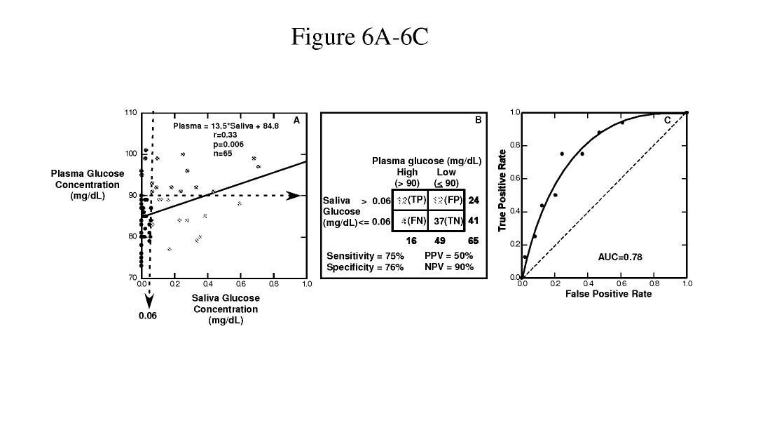

Recognition that salivary glucose exhibits a threshold response can aid in the interpretation of the diagnostic potential of salivary glucose. First, it means that salivary glucose levels are likely useful for diagnosis of high glucose conditions. As such, salivary glucose could still prove a useful indicator of pathological status—a lack of salivary glucose would be good news for the patient with diabetes! Yet, the issue of false negatives has to be considered. In the present study, many readings occurred along the axis of zero for salivary glucose concentration (FIG. 6A), which may represent children with a higher-than-average salivary glucose concentration threshold which had not yet been exceeded. It is commonly reported in the salivary glucose literature that there is a significant correlation between plasma and salivary glucose levels under conditions in which hyperglycemia is expected, such as in subjects with uncontrolled diabetes, but that there is little or no correlation under conditions where hyperglycemia may not be expected, such as in healthy subjects and in patients with controlled diabetes. This fact becomes understandable if blood glucose levels are at or below the value of the salivary glucose concentration threshold. Second, it is unknown what parameters modulate the salivary glucose concentration threshold in humans. The original research in dogs showed that an intravenous infusion of insulin increased the threshold level for the salivary glucose concentration. Whatever mechanisms control the salivary glucose concentration threshold, it is likely that salivary glucose concentration results from simple passive diffusion from plasma.

The recognition of threshold kinetics in salivary glucose concentration measurements suggests that if children have measurable salivary glucose levels ≧0.06 mg/dL, it is likely that they have high plasma glucose levels. If they have salivary glucose levels above 1 mg/dL, then they are likely hyperglycemic (plasma levels >100 mg/dL), although this criterion could be greatly modified in certain defined clinical conditions and carries a low positive predictive value (PPV=50%). As a screening diagnostic, however, low positive predictive value is acceptable, since the test would ultimately be used as a trigger to seek professional confirmation. Indeed, a high false-negative rate, which is not the case here, would be the worst case scenario, since it would inappropriately assure the absence of high plasma glucose levels. In the present study, the probability that a child would not have a high plasma glucose level if the saliva glucose is low (≦0.06 mg/dL), the negative predictive value, was 90%. Although the present methods were designed for research purposes, by this analysis, saliva glucose appears to have reasonable characteristics to serve as a screening diagnostic for high plasma glucose in children.

TABLE 7
Representative studies that provide analytical data for evaluating the relationship between salivary glucose and blood glucose
concentrations as a threshold response. One study of urine glucose concentration is also included for comparison.

| | | | | Threshold | | | | |
|---|---|---|---|---|---|---|---|---|
| Study | | Species | Sample | (mg/dL) | Slope | r | p | Population |
| Forbat et al. (1981)²⁵ | | Human | Parotid saliva^{a} | 136.8 | 6.4 | 0.20 | 0.38 | 20 diabetic adults^{b} |
| Amer et al. (2001)¹⁵ | | Human | Whole saliva | 107.0 | 12.7 | 0.78 | <0.05 | 135 diabetic adults |
| Abikshyeet et al. (2012)¹⁴ | | Human | Whole saliva | 99.7 | 13.0 | 0.77 | <0.01 | 106 diabetic adults |
| Current study | | Human | Whole saliva | 84.8 | 13.5 | 0.33 | 0.006 | 65, 11-year-old children |
| Yamaguchi et al. | A | Human | Submaxillary + | 110 | 22.4 | | | 6 healthy adults (A-F)^{c} |
| (1998)²⁶ | B | | sublingual | 68 | 87.1 | | | |
| | C | | saliva | 104 | 13.8 | | | |
| | D | | | 60 | 111.1 | | | |
| | E | | | 105 | 46.7 | | | |
| | F | | | 88 | 51.0 | | | |

| | | | | | | |
|---|---|---|---|---|---|---|
| Average | | | 89.1 ± 8.6 | 55.3 ± 15.3 | | |
| Hayford et al. (1983)²⁷ | Human | Urine | 152.7 | 47.4 | 0.80 | 24 diabetic adults |
| Langley et al. (1958)²⁴ | Dogs | Parotid saliva | 512.0 | | | 10-12 kg dogs |

^{a}Stimulated with lemon juice and parotid massage
^{b}50 μL samples only
^{c}75 g oral glucose tolerance test

Metabolic syndrome in childhood predicts the development of cardiovascular disease and Type 2 diabetes in adulthood. Testing for features of metabolic syndrome, such as fasting plasma glucose concentration, requires blood sampling, which can be difficult in children. Salivary glucose concentration as a surrogate measurement for plasma glucose concentration were evaluated in 11-year-old US children. Children from Portland, Me. and Cambridge, Mass. with a mean age of 10.6±0.2 years old, with obese and overweight children being slightly older provided 6-hour fasting samples of both blood and whole saliva. Salivary glucose levels were measured with a high-sensitivity assay (sensitivity=0.002 mg/dL). Plasma glucose levels were determined by a commercial clinical laboratory. Blood pressure, salivary flow rate, height, and weight were also measured. A total of 65 children were enrolled, of which 63% were male (see Table 8). There were two underweight children (3.1%), 30 normal-weight children (46.2%), 12 overweight children (18.4%), and 21 obese children (32.3%). The number of underweight children in the group was too small to be meaningful for statistical analyses. Both diastolic and systolic blood pressure tended to be higher in obese children relative to normal-weight children (124/69 mm Hg vs. 116/67 mm Hg), but neither value varied significantly. The mean overall fasting plasma glucose level was 86.3 mg/dL, and did not differ significantly between body-weight groups.

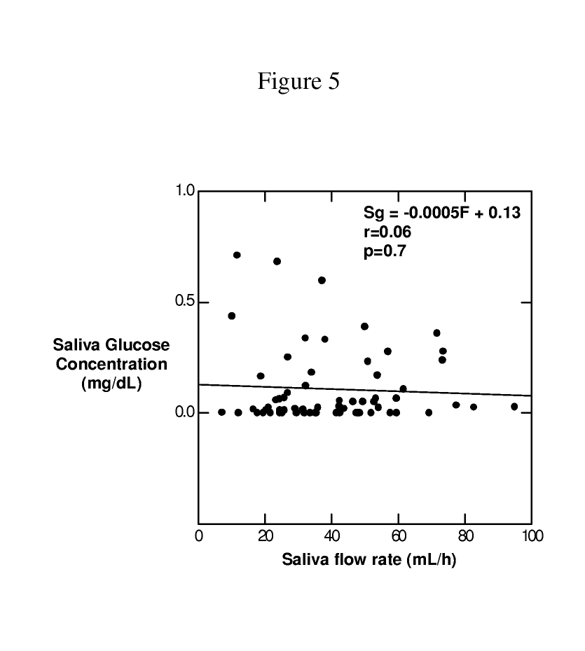

While no significant functional correlation was noted between salivary glucose concentrations and saliva flow rate, salivary glucose levels were still insightful. The mean saliva collection time was 7.68±4.8 min (range, 2-28 min). The average volume collected was 3.93±0.92 mL (range, 3-8 mL). The mean overall fasting salivary glucose level was 0.11±0.02 mg/dL, and the mean salivary glucose excretion rate was 41.2±8 μg/h, with no statistically significant differences seen between children in the different body weight categories. The salivary flow rate was 40.1±19.3 ml/h (range 7-95 ml/h), which was not statistically different between body weight categories. By regression analysis, the saliva glucose concentration did not appear to be functionally related to saliva flow rate (FIG. 5).

TABLE 8
Population characteristics of the enrolled children. Tabulated ranges are mean ± SEM.
Overall differences between body weight categories were tested by ANOVA. Significant pairwise
differences were determined by post hoc analysis using Tukey's honestly-significant difference
test. Values with the same superscript letter within each row differed at p < 0.05.

| | Body Weight Category | | | | |
|---|---|---|---|---|---|
| | Underweight | Normal Weight | Overweight | Obese | Overall |
| N | 2 | 30 | 12 | 21 | 65 |
| Age (y) | 9.8 ± 1.4 | 10.0 ± 0.2^{a} | 11.2 ± 0.5 | 11.2 ± 0.4^{a} | 10.6 ± 0.2* |
| Sex [number of males (%)] | 1 (50) | 18 (60) | 7 (58.3) | 15 (71.4) | 41 (63.0) |
| Plasma glucose (mg/dL) | 89.5 ± 10.5 | 86.5 ± 1.2 | 84.8 ± 1.9 | 86.5 ± 1.5 | 86.3 ± 0.8 |
| Salivary glucose (mg/dL) | 0.29 ± 0.04 | 0.10 ± 0.02 | 0.11 ± 0.03 | 0.09 ± 0.04 | 0.11 ± 0.02 |
| Glucose excretion (μg/h) | 88.1 ± 20.4 | 42.6 ± 10.9 | 54.3 ± 24.6 | 27.9 ± 13.7 | 41.2 ± 8.0 |
| Saliva flow rate (mL/h) | 29.6 ± 2.6 | 40.5 ± 3.4 | 44.2 ± 5.5 | 38.3 ± 4.8 | 40.1 ± 2.4 |
| Diastolic blood pressure (mm Hg) | 69 ± 2 | 67 ± 1 | 68 ± 2 | 69 ± 2 | 69 ± 1 |
| Systolic blood pressure (mm Hg) | 119 ± 2 | 116 ± 2 | 121 ± 3 | 124 ± 2 | 120 ± 1 |
| BMI | 13.5 ± 0.3^{ab} | 17.2 ± 0.3^{cd} | 22.2 ± 0.6^{ace} | 28.5 ± 1.2^{bde} | 21.7 ± 0.8* |

BMI, body mass index
*model p < 0.05
Underweight subjects (13.5 =^{ab}) were significantly different than the overweight subjects (22.2 =^{a}) and were also significantly different than the obese subjects (28.5 =^{b}).
Normal weight subjects (17.2 =^{cd}) were significantly different than overweight (22.2 =^{c}) and obese subjects (28.5 =^{d}).
Overweight subjects (22.2 =^{ace}) were significantly different than underweight (^{a}), normal weight (^{c}) and obese subjects (^{e}).
Obese (^{bde}) subjects were significantly different than underweight (^{b}), normal weight (^{d}) and overweight (^{e}) subjects.
Values in Table 8 with the same superscript letter within each row differed at p < 0.05.

Salivary glucose levels exhibited threshold kinetics. There was a significant association between plasma and salivary glucose levels, although there was considerable variability (FIG. 6A). The functional relationship between the plasma and salivary glucose concentrations clearly exhibited a threshold. Plasma glucose concentrations less than the threshold value of 84.8 mg/dL produced unmeasurable salivary glucose levels. At plasma concentrations greater than 84.8 mg/dL, the plasma glucose concentration increased at a rate 13.5 times the saliva glucose concentration. If positive salivary glucose values occur only at plasma values greater than zero, a threshold is implied. Mathematically, this occurs when the y-intercept assumes a large value rather than zero.

Salivary glucose levels had a high negative predictive value. The diagnostic potential of salivary glucose to predict plasma glucose is illustrated in FIG. 6B. Diagnostic sensitivity and specificity were 75% and 76%, respectively, but the false-positive rate was much higher than the false-negative rate, and the false-positive rate was equal to the true-positive rate. As such, salivary glucose levels would correctly identify at least about 50% of children with high plasma glucose levels. Conversely, the false-negative rate was small relative to the true-negative rate, so the probability of a child having a high plasma glucose level with a low salivary glucose level (<0.06 mg/dL) is very low, and the negative predictive value is high (90%). FIG. 6C illustrates the receiver operating curve (ROC) with an area under the curve measurement of 0.78 an indication of good accuracy of the salivary glucose method.

The results provided herein above were carried out using the following methods and materials.

Subject Selection

Children participating in the study were in either the 4^{th} or 5^{th} grades and had prior parental/guardian signed informed consent. Assent from the children was obtained the day of the visit. The study protocol and informed consent for the US children were reviewed and approved by the Forsyth Institutional Review Board in Cambridge Mass., U.S.A. Protocol and informed consent documents for the Kuwaiti children were reviewed and approved by the Dasman Diabetes Institute Ethical Review Committee in Kuwait City, Kuwait.

Data Collection

Data and saliva samples were collected from 8,319 children during 182 visits to 39 Kuwaiti schools between Oct. 2, 2011 and May 15, 2012. At the time of entry into the study, each subject was identified by a number representing the collection date plus a unique number for that subject on that day. Subject identification, height, weight, blood pressure, food preferences, oral examination, fitness and sleep parameters were collected and entered into a programmed iPad (Apple corp., Cupertino Calif.) system for internet transfer. Fitness was measured by heart rate elevation (beats/minute) following a standard 3-minute exercise. Body weight categories were defined using a BMI z-score based on 2000 Centers for Disease Control and Prevention growth charts. As a separate validation, blood and saliva samples were collected between Feb. 23, 2011 and Sep. 23, 2011 from 53 children living in Maine and in Massachusetts. Blood samples (10 ml) were taken from the median cubital vein. Otherwise the protocol was identical to that of the Kuwaiti protocol.

The results reported herein above were obtained using the following methods and materials.

Saliva Collection

All saliva samples were collected as whole saliva by expectoration of approximately 3 ml after a 15 ml water rinse. Samples were collected starting at 8:30 in the morning before the children were given breakfast. Samples were maintained on ice for transport to laboratories at the Dasman Institute. The samples were centrifuged at 4° C. One milliliter of each supernatant was transferred to a screw-cap 2D barcoded storage tube (Thermo Scientific), read by an electronic barcode reader (Thermo Scientific VisionMate ST), and the number transferred, along with the subject number, to a computer spreadsheet. The sample vials were sealed by a torque-controlled tube capper (Thermo Scientific 8-Channel Screw Cap Tube Capper), placed in a 96-vial rack (Thermo Scientific Latch Rack) and frozen at −80° C. Racks were air-transferred from Kuwait under temperature monitored dry ice (Biocair, Boston Mass.) to the Forsyth Institute and maintained at −80° C. until assayed (average time to assay=0.88±0.06 y). Upon receipt, 2D barcodes were read on each rack and double-checked against recorded values in the original spreadsheet.

From the 8,319 samples, 744 samples were randomly selected for assay so that 93 were from children of each of the defined body weight categories described in the data analysis section (underweight, normal weight, overweight and obese) for each sex. The children were divided into 8 groupings (2 sexes times 4 body weight categories). Randomized selection of individuals from each grouping was achieved by applying a random number between 1 and x=integer(total number in each group/93) to each child, sorting for all assigned the number 1 and selecting the first 93. Normal, overweight and obese categories were represented by equal numbers of saliva samples (93 each) from male and female subjects. Underweight children were included as a group so that biomarkers were identified that increase with increasing obesity throughout the weight range of children being evaluated. Due to a limited numbers of male underweight subjects available, 64 male and 122 female subjects were selected for that category.

Multiplex Analysis of Salivary Biomarkers

All assays were run with four multiplex magnetic bead panels using a Luminex 200 (Luminex Corp, Austin Tex.). Software used to evaluate the results was Bio-Plex Manager, (Version 5.0; Bio-Rad Laboratories, Inc., Hercules, Calif.). Four multiplex immunoassay panels were used to evaluate 744 Kuwaiti saliva samples for 20 bioactive molecules. Interferon gamma (IFN-γ), interleukin-10 (IL-10), interleukin-12P70 (IL-12P70), interleukin-13 (IL-13), interleukin-17A (IL-17A), interleukin-1β (IL-1β, interleukin-4 (IL-4), interleukin-6 (IL-6), interleukin-8 (IL-8), monocyte chemotactic protein-1 (MCP-1), tumor necrosis factor-α (TNF-α) and vascular endothelial growth factor (VEGF) were measured by a 12-plex human cytokine/chemokine panel with no dilution (Millipore cat #HCYTOMAG-60K; lot #2055690). Metabolic hormones, ghrelin, insulin and leptin, were measured by a 3-plex human metabolic hormone panel with no sample dilution (Millipore cat #HMHMAG-34AK; lot #2055724). Myeloperoxidase (MPO) and matrix metalloprotein 9 (MMP9) were measured by a 2-plex human cardiovascular disease panel with 1:2 sample dilution (Millipore cat #HCVD1-67AK; lot #2055723). Adiponectin, CRP and resistin were measured in a 3-plex human obesity panel with 1:2 sample dilution (R&D cat #LOB000, LOB 1065, LOB 1707, LOB 1359; lot #300710). The panel used to evaluate insulin in blood and saliva of U.S. children was a human metabolic hormone panel single plex for insulin (Millipore cat #HMHMAG-34K-01EMD) with no dilution and following manufacturer's protocol. All assays were performed following manufacturers' protocol, with the exception of an additional 3 standards to increase the range of detection. A more detailed examination quality control and validation has been described elsewhere (PloS one, 2013; 8:e59498).

Saliva samples were thawed at 4° C. overnight prior to assaying and kept on ice throughout the assay procedures. Manufacturers' protocols were followed for all four panels, with a general protocol as follows: All kit components were brought to room temperature. Reagents were prepared as per kit instructions (wash buffers, beads, standards, etc.). Assay plates (96-well) were loaded with assay buffer, standards, samples, and beads and then covered and incubated on plate shaker (500 rpm) for either 3 hours at room temperature for the obesity panel, or overnight at 4° C. After primary incubation, plates were washed three times and then detection antibody cocktail was added to all wells; the plates were covered and left to incubate at room temperature for 1 hour on plate shaker. After the one hour incubation, streptavidin-phycoerythrin fluorescent reporter was added to all wells, and then the plate was covered and incubated for 30 minutes at room temperature on plate shaker. Plates were then washed three times and beads were resuspended in wash buffer, placed on shaker for 5 minutes, and then read on Luminex platform following manufacturers' specifications and using Bio-Plex Manager software version 5.0.

Data Analysis

Body weight categories were defined on BMI z-score based on published growth charts. By this criterion, obese was equal to or greater than the 95^{th} percentile, overweight was between the 85^{th} and 95^{th} percentile, normal healthy weight was between the 5th and 85^{th} percentile and underweight was less than the 5^{th} percentile. CDC software was used for this purpose. The multiplex probe data was clearly not normally distributed so that conventional parametric analysis could introduce bias. To account for the non-normality of multiprobe data, a multivariate rank-based Wilcoxon regression method was applied. Age (years) and sex (1=male, 0=female) were adjusted when evaluating the relationship between the probe concentration and body weight categories. Values for age, BMI, waist circumference, and systemic blood pressure were analyzed for significance between body weight categories by a Kruskal-Wallis rank sum test followed by pairwise comparisons using the Wilcoxon rank sum test. Selection of predictor variables, diagnostic sensitivity and specificity was determined by CART software (Salford Systems, San Diego Calif.). Linear, semi-logarithmic and log-log regression models were tested for the distribution of their residuals before selecting the log transformation of both insulin measurements for analysis. Analysis of predictor variable categorical comparisons was by the Mann-Whitney U test. P-values were computed and a Bonferroni correction of 0.0004 (for overall p<0.05) applied to control the familywise error rate associated with 125 test procedures.

Patients

Children of both sexes between the ages of 10 and 11 years were recruited by advertisement in the Cambridge, Mass. and Portland, Me. areas from February 2011 to September 2011 YEAR using a protocol reviewed and approved by the Forsyth Institutional Review Board. Only children with extreme disease conditions, such as immuno-deficiencies, cancer, or serious behavioral disorders were excluded. The target study size was set at 75 children based on obtaining approximately 25 children in each of the body weight categories of normal, overweight and obese. Both informed consent and participant assent were obtained from the parents/guardians of each child and from the child.

Assessments

All assessments were conducted by trained examiners. Height was measured by stadiometer and weight was measured by a calibrated bathroom scale. Blood pressure and heart rate were measured after the children had sat quietly for 10 minutes with both feet on the floor, and the measurements were performed using an automated wrist monitor sized appropriately for children. Fitness was measured by a standardized 3-minute step test (Suriano et al., J. Pediatr. October 2010; 157(4):552-558.) where the change in heart rate was measured by a pulse oximetry probe (RAD-57, Masimo Corporation, Irvine, Calif.) applied to the finger.

Saliva Collection

Saliva was collected under 6-hour fasting conditions by dental hygienists. Each child was given a wrist label with a printed number and a dated, labeled, sterile, 15-mL plastic screw-top centrifuge tube (Product #430791, Corning Incorporated Life Sciences, Tewksbury, Mass.) with the same number as the wrist band. Before saliva collection, each child rinsed with and swallowed 15 mL of water. Whole saliva (approximately 3 mL) was collected by having the child drool, not spit, into the screw-top tube. Tubes were kept on ice while saliva was allowed to accumulate in the child's mouth. A monitor recorded the start time of the saliva collection, verified that 3 mL was collected from each child, recorded the stop time for each child, assured that the screw cap was securely applied to the tube, and transferred the labeled tube to an ice bath for temporary storage. The average collection time was 8.1±0.7 minutes (mean±S.E.).

Salivary Glucose Analysis

Saliva samples were weighed and then centrifuged at 2,800 RPM at 4° C. for 20 minutes. Two 1-mL aliquots of the supernatants were transferred to screw-cap storage tubes and maintained frozen at −80° C. until assayed. For the glucose analysis, the glucose oxidase method using fluorescent emission of the dye (Glucose Colorimetric/Fluorometric Assay Kit #K606-100, BioVision, Inc, Mountain View, Calif., USA) measured at Ex/Em −535/590 nm was adapted to work on a Tecan Freedom EVO® 150 robotic processor with an 8-channel liquid handling arm (Tecan Group Ltd, Männedorf, Switzerland). The fluorescence was measured by a spectrophotometer (Infinite® 200 Pro, Tecan Group Ltd, Männedorf, Switzerland) using reverse 96-well plate reading mode. The 3 sigma detection limit of the glucose assay was 0.002 mg/dL. 30 μL of saliva supernatant were assayed for each sample. Standards of 0.12, 0.24, 0.48 and 0.96 mg/dL were assayed in triplicate on each run. Coefficient of variation (CV) measurements on the same day and over a period of 98 days are found in Table 9. These data indicate that variation is increased for more dilute samples and ranges from 23 to 101 depending on glucose concentration. CV values reported for saliva sample analysis are often higher than those reported for serum analysis of the same biochemical (Browne et al., PloS one. 2013; 8(4):e59498) possibly due to saliva characteristics unique to saliva that increase measurement variability (viscosity, propensity to form bubbles, etc.).

TABLE 9
Within and between day coefficient of variation
measurements of glucose in saliva samples

| Glucose Concentration | | |
|---|---|---|
| (mg/dL) | Within-day CV^{a} | Between-day CV^{b} |
| ≧0.2 | 22.9 | 27.3 |
| 0.2 < concentration > 0.1 | 54.6 | 41.0 |
| ≦0.1 | 101.1 | 46.1 |

^{a}Duplicate measurements on one day (18 samples)
^{b}Three measurements over 98 days (47 samples)

Plasma Glucose analysis

Two milliliters of blood were obtained by venipuncture from the antecubital fossa under 6-hour fasting conditions and were collected into potassium oxalate/sodium fluoride (grey top) tubes. Collected blood samples were centrifuged at 2,800 RPM for 20 min. Two 0.5-mL plasma aliquots were dispensed into labeled, sterile, 1.8-mL screw-cap vials (Nunc® CryoTubes® #363401, Thermo Fisher Scientific Inc., Asheville, N.C., USA) which were stored at −80° C. until assayed. Glucose plasma levels were measured by a commercial clinical laboratory (test cod 484, Quest Diagnostics Inc, Cambridge, Mass., USA).

Statistical and Analytical

BMI was calculated by dividing body weight in kilograms by height in meters squared. Body weight categories were determined from BMI percentile using The Centers for Disease Control Software as follows: underweight, <5th percentile; normal weight, 5th-84th percentile; overweight, 85th-94th percentile; obese >95th percentile (CDC. Child and Teen BMI Calculator. 2012). The saliva flow rate was computed by dividing the tarred weight of the saliva collection tube by the difference in the start and stop collection times in h. The salivary glucose excretion rate was calculated by multiplying the salivary flow rate by the saliva glucose concentration. Associations between saliva glucose values and plasma values were investigated through linear regression analyses. The analysis of significant differences between parameters related to population characteristics was by analysis of variance (ANOVA). Significant pairwise differences were determined by post hoc analysis using Tukey's honestly-significant difference test.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of characterizing a subject as having or at risk of developing metabolic syndrome, the method comprising detecting an alteration in the level of one or more marker selected from the group consisting of C-reactive protein (CRP), insulin, glucose, leptin, and adiponectin in a saliva sample of the subject relative to a reference, thereby characterizing the subject as having or at risk of developing a metabolic disease.

2. A method of detecting inflammatory obesity or a propensity to develop inflammatory obesity in a subject, the method comprising detecting an alteration in the level of a marker selected from the group consisting of CRP, insulin, glucose, IL-6, IL-10, resistin, IL-1beta, MMP-9, and adiponectin in a saliva sample from the subject relative to a reference, thereby detecting inflammatory obesity or a propensity to develop inflammatory obesity in the subject.

3. A method of identifying a non-obese subject as having or having a propensity to develop a metabolic syndrome or inflammatory obesity, the method comprising detecting an alteration in the level of a marker selected from the group consisting of CRP, insulin, glucose, IL-6, IL-10, resistin, IL-1beta, MMP-9, and adiponectin relative to a reference; and identifying the subject as having or having a propensity to develop a metabolic disease or inflammatory obesity.

4. The method of claim 1, wherein the level of adiponectin is decreased relative to the reference;

5. (canceled)

6. The method of claim 1, wherein the level of insulin, leptin, C-reactive protein (CRP), or glucose, is increased relative to a reference.

7-11. (canceled)

12. The method of claim 1, wherein the increase in salivary glucose level indicates a high plasma glucose level.

13-14. (canceled)

15. The method of claim 1, wherein the subject is underweight, normal healthy weight, overweight or obese.

16. The method of claim 1, further comprising comparing clinical measurements of the subject relative to the reference.

21. The method of claim 1, wherein the alteration in polypeptide level is detected by Western blot, enzyme-linked immunoassay, direct immunoassay, radiometric assay, fluorescence, or protein activity.

22-24. (canceled)

25. The method of claim 3, wherein the non-obese subject is identified as having increased levels of glucose, CRP, insulin, IL-6, IL-10, resistin, IL-1b, and MMP-9.

26. The method of claim 2, wherein increased levels of salivary insulin, glucose, and CRP are indicative of inflammatory obesity or a propensity to develop inflammatory obesity.

27. The method of claim 2, wherein the increased levels of salivary insulin levels, but reduced adiponectin levels are indicative of non-inflammatory obesity.

28. The method of claim 1, further comprising measuring a biomarker selected from the group consisting of Adiponectin, Resistin, IL-8, VEGF, MCP-1, CRP, Insulin, IL-1β, glucose, MPO, MMP-9, IL-12P70, IL-4, IL-6, IL-13, TNF-alpha, IL-10, IFN-gamma, Leptin, Ghrelin, and IL-17A.

29. The method of claim 1, further comprising identifying a subject as in need of therapeutic intervention to prevent or treat a metabolic disease, wherein the method comprising detection of increased levels of C-reactive protein (CRP), insulin, glucose, leptin and reduced levels of adiponectin to identify the subject as in need of therapeutic intervention to prevent or treat the metabolic disease.

30. The method of claim 29, wherein the therapeutic intervention is selected from the group consisting of dietary restriction, increased exercise, or treatment with an anti-inflammatory agent.

31. A biomarker panel comprising C-reactive protein (CRP), insulin, leptin, glucose, and adiponectin; or CRP, insulin, glucose, IL-6, IL-10, resistin, IL-1beta, MMP-9, and adiponectin; or capture molecules that specifically bind said biomarkers.

32. The biomarker panel of claim 31, further comprising a biomarker selected from the group consisting of Resistin, IL-8, VEGF, MCP-1, IL-1β, MPO, MMP-9, IL-12P70, IL-4, IL-6, IL-13, TNF-alpha, IL-10, IFN-gamma, Ghrelin, and IL-17A.

33. (canceled)

34. A lateral flow device comprising a liquid permeable material defining the following portions in capillary communication:
a) a first portion that is the site for application of a saliva sample, comprising a liquid permeable medium, an analyte-binding conjugate that binds an analyte selected from the markers in a panel of claim 31 and a control conjugate;
b) a second portion comprising a liquid permeable medium; and
c) a third portion that is the site for detecting the binding of the analyte-binding conjugate at the test site and the binding of the control conjugate at a control site, the third portion comprising a liquid permeable medium having the analyte fixed to the medium at the test site, and having a control conjugate binder present at a control site.

35. A method for characterizing a subject as having or at risk of developing metabolic syndrome, the method comprising contacting the panel of claim 31 with a salivary sample of the subject, and detecting binding.
